(19) United States
(12) Patent Application Publication
(10) Pub. No.: US 2015/0064713 A1
(43) Pub. Date: Mar. 5, 2015

(54) METHODS, KITS AND MEANS FOR DETERMINING INTRACELLULAR INTERACTIONS

(71) Applicants: Philippe Bastiaens, Dortmund (DE); Stephanie Reisewitz, Dortmund (DE); Giuseppe Arrabito, Dortmund (DE); Leif Dehmelt, Dortmund (DE); Silke Gandor, Dortmund (DE); Hendrik Schroeder, Dortmund (DE); Christof M. Niemeyer, Dortmund (DE)

(72) Inventors: Philippe Bastiaens, Dortmund (DE); Stephanie Reisewitz, Dortmund (DE); Giuseppe Arrabito, Dortmund (DE); Leif Dehmelt, Dortmund (DE); Silke Gandor, Dortmund (DE); Hendrik Schroeder, Dortmund (DE); Christof M. Niemeyer, Dortmund (DE)

(21) Appl. No.: 14/016,953

(22) Filed: Sep. 3, 2013

(51) Int. Cl. G01N 33/566; C07K 14/00

(52) U.S. Cl. CPC G01N 33/566; C07K 14/00
USPC 435/7.2; 530/350

(57) ABSTRACT

Methods, kits and systems for determining whether a reaction occurs between a chimeric transmembrane receptor and an intracellular interaction partner thereof within a cell.

### TECHNICAL FIELD

The present invention relates to methods, kits and systems for determining whether a reaction occurs between a chimeric transmembrane receptor and an intracellular interaction partner thereof within a cell.

### BACKGROUND

Current methods to study protein reactions, such as their interactions are either limited to lysed cells, or fail to provide for multiplex measurements of several interactions in an individual living cell (Peyker A, Rocks O, Bastiaens P I. Chembiochem. 6:78). General, extendable methods to determine multiple protein interactions at the same time in individual living cells are not available. Current approaches to measure protein reactions in cells, such as those using FRET or BRET, are not extendable to measure more than two protein reactions in parallel in individual living cells. Schwarzenbacher et al. describes the measurement of interactions between a native receptor, CD4, and one of it's binding partners, Lck (Schwarzenbacher et al. (2008) Nature methods 5, 1053-1060). The approach used in this publication, however, is limited to measuring a single interaction in an individual cell. In Shen et al., two co-stimulatory ligands were immobilized and the effect of their spatial organization on cell behavior and interactions with known binding partners of the cognate receptors was studied (Shen et al. (2008) Proc Natl Acad Sci USA 105, 7791-7796). In another study (Zamir et al. (2010) Nature methods 7, 295-298), the interaction between “bait”-fused quantum dots and a soluble, fluorescently labeled “prey” was reported. The technical problem underlying the present invention was to identify an extendable strategy for studying multiple intracellular molecular reactions in parallel in an individual living cell. The solution to this technical problem is achieved by providing the embodiments characterized in the claims.

### SUMMARY

Accordingly, the present invention relates in a first embodiment to a method for determining whether a reaction occurs between a chimeric transmembrane receptor and an intracellular interaction partner thereof within a cell, said method comprising the steps of: a. providing a cell comprising: i. at least two distinct chimeric transmembrane receptors each comprising: (a) an extracellular binding domain, (b) a transmembrane domain, and (c) an intracellular domain, wherein said at least two transmembrane receptors are distinct in that (i) at least two of the domains (a), (b) and (c) are of different origin, (ii′) in that said extracellular binding domain of each of said at least two transmembrane receptors specifically interacts with a different extracellular compound, and (iii′) in that said intracellular domain of each of said at least two transmembrane receptors is different; and ii. one or more different potential intracellular interaction partners that (i′) for step b.i. or step b.ii.(i′) or (ii′) are labelled with first labels; or (ii′) for step b.ii.(iii′) are unlabelled; b. contacting the cell with at least two different extracellular compounds, wherein each of said at least two extracellular compounds is bound to a surface i. in different areas of the same support, and/or ii. on different supports, (i′) wherein each support and its cognate transmembrane receptor form a complex that is labelled with a second label, (ii′) wherein each support can be distinguished by its shape and/or size, and/or (iii′) wherein in each support and its cognate transmembrane receptor the intracellular domain is labelled with a label or a pair of labels which is capable to indicate reactions with one or more potential intracellular interaction partners of step a.ii. with the intracellular domain; and c. detecting i. said first label in step b.i.; and/or ii. said first label and second label, shape and/or size in step b.ii.; wherein i. for step c.i. the presence of a signal of said first label in (an) area(s) comprising the cognate extracellular compound and ii. for step c.ii.(i′) the presence of co-localized signals of said first and second label(s), for step c.ii.(ii′) co-localization of said first signal with said support, and for step c.ii.(iii′) a detectable conformational change of the label or a detectable energy transfer between the pair of labels is indicative of a reaction between a potential intracellular interaction partner with a distinct chimeric transmembrane receptor.

### BRIEF DESCRIPTION OF THE FIGURES

FIG. 1 is a schematic illustration of the principle of live cell multiplex biosensors.

FIG. 2 is a schematic illustration of bait presenting artificial receptor constructs (bait-PARCs) and immobilized antibodies.

FIG. 3 is (a) a Schematic for the design of two orthogonal artificial receptor-extracellular compound pairs based on the zinc-finger DNA interaction; and b) microscopic analysis of specific oligonucleotide binding to cells expressing the receptor variant 2.

FIG. 4 is (a) a schematic of the application of DNA-directed immobilization (DDI) to generate arrays of immobilized antibodies; (b) micrographs of immunohistochemical assays illustrating bait-PARCs displaying VSVG epitope tags recruited to anti-VSVG functionalized surface patterns within the plasma membrane of COS7 cells; (c) a micrograph showing selective surface functionalization via DDI; (d) micrographs of checkerboard patterns of two distinct antibodies, anti-VSVG and anti-HA, generated via DDI and identified based on intensity coding of Atto 740 fluorophores.

FIG. 5 is a) a schematic for micro-patterning of receptors in living cells via surface-immobilized submicrometer size streptavidin-functionalized beads; b) Total internal reflection micrographs of recruitment of a kinase-dead growth factor receptor to surface immobilized beads in living cells; and (c) micrographs illustrating rRecruitment of growth factor receptors to mobile beads in living cells.

FIG. 6 is. a) a schematic of the surface modification procedure for microstructuring by immobilization of DNA oligonucleotides on a glass surface via photolithography; and b) a fluorescent micrograph of Alexa-488 and Alexa-568 labeled oligonucleotides, which interact with sequentially written complementary oligonucleotides (structure size: approx. 2 μm).

FIG. 7 is a schematic illustration of indirect coupling via secondary extracellular compounds involving DDI.

FIG. 8 is a) a schematic of domain structures of bait-PARCs to measure PKA subunit interactions; b) (LEFT) micrographs illustrating recruitment of cytosolic prey protein mCherry-cat-α to bait microstructures containing the regulatory domain RII-β of PKA before and after pharmacological perturbation and (RIGHT) a chart of derived prey recruitment kinetics; c) (LEFT) Image of a representative experiment involving two distinct regulatory domains on bait-PARCs co-expressed together with the prey protein mCherry-cat-α depicting cells grown on a DNA-immobilized antibody array, and (RIGHT) total internal interference reflection micrographs showing the recruitment of prey proteins to the two distinct bait proteins during pharmacological perturbation.

FIG. 9 is a) a graph of paired measurements of the interaction between the prey protein and the two bait proteins in individual, resting cells; and b) a chart of temporal cross-correlation profiles for the response of the two distinct regulatory subunits during β-adrenergic receptor stimulation.

### DETAILED DESCRIPTION

In this specification, a number of documents including patent applications and manufacturer's manuals is cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Accordingly, the present invention relates in a first embodiment to a method for determining whether a reaction occurs between a chimeric transmembrane receptor and an intracellular interaction partner thereof within a cell, said method comprising the steps of: a. providing a cell comprising: i. at least two distinct chimeric transmembrane receptors each comprising: (a) an extracellular binding domain, (b) a transmembrane domain, and (c) an intracellular domain, wherein said at least two transmembrane receptors are distinct in that (i) at least two of the domains (a), (b) and (c) are of different origin, (ii′) in that said extracellular binding domain of each of said at least two transmembrane receptors specifically interacts with a different extracellular compound, and (iii′) in that said intracellular domain of each of said at least two transmembrane receptors is different; and ii. one or more different potential intracellular interaction partners that (i′) for step b.i. or step b.ii.(i′) or (ii′) are labelled with first labels; or (ii′) for step b.ii.(iii′) are unlabelled; b. contacting the cell with at least two different extracellular compounds, wherein each of said at least two extracellular compounds is bound to a surface i. in different areas of the same support, and/or ii. on different supports, (i′) wherein each support and its cognate transmembrane receptor form a complex that is labelled with a second label, (ii′) wherein each support can be distinguished by its shape and/or size, and/or (iii′) wherein in each support and its cognate transmembrane receptor the intracellular domain is labelled with a label or a pair of labels which is capable to indicate reactions with one or more potential intracellular interaction partners of step a.ii. with the intracellular domain; and c. detecting i. said first label in step b.i.; and/or ii. said first label and second label, shape and/or size in step b.ii.; wherein i. for step c.i. the presence of a signal of said first label in (an) area(s) comprising the cognate extracellular compound and ii. for step c.ii.(i′) the presence of co-localized signals of said first and second label(s), for step c.ii.(ii′) co-localization of said first signal with said support, and for step c.ii.(iii′) a detectable conformational change of the label or a detectable energy transfer between the pair of labels is indicative of a reaction between a potential intracellular interaction partner with a distinct chimeric transmembrane receptor.

In step b. it is preferred that each of the at least two extracellular compounds specifically interacts with a different transmembrane receptor of said at least two transmembrane receptors.

A “reaction” as used herein refers to a detectable response of the chimeric transmembrane receptor triggered by the intracellular interaction partner. The reaction may either represent a stable or transient direct interaction between the chimeric transmembrane receptor and the intracellular interaction partner or a chemical modification of the chimeric transmembrane receptor by the intracellular interaction partner. Throughout this specification a reaction is preferably an interaction as further defined herein below. It follows that the potential intracellular interaction partner as defined herein below is preferably a potential intracellular binding partner throughout this specification.

The term “determining whether a reaction occurs” has the established meaning in the art and extends to determining presence or absence of a detectable response of the chimeric transmembrane receptor triggered by the intracellular interaction partner. The reaction may be a previously known or unknown reaction. The reaction may be quantified. The method according to the invention also extends to observing a reaction, wherein said observing may also include observing or monitoring over time and/or at more than one location, preferably locations within the cytoplasm or at the inner surface of the plasma membrane within a given cell. Such quantifying as well as monitoring in space and/or over time is the subject of preferred embodiments discussed further below.

A “potential intracellular interaction partner” according to the invention may be any molecule or complex of the same or different molecules and may or may not be capable of triggering a reaction within the intracellular domain of a chimeric transmembrane receptor. In other words, the term “potential intracellular interaction partner” in its broadest form embraces candidate reaction partners. For example, the capability of a given potential intracellular interaction partner as candidate partner to react with a variety of different intracellular domains of different chimeric transmembrane receptors can be tested. As specified above, the potential intracellular interaction partner is preferably a potential intracellular binding partner throughout this specification.

The choice of the potential intracellular interaction, preferably binding partners of the invention depends on the specific problem to be addressed in that intracellular domains of the chimeric transmembrane receptors have to be chosen that are known or are not known to be reactive with said potential intracellular interaction, preferably binding partner. For example, if the reaction of the partners of a known reaction pair is to be determined, monitored or quantified in dependency of the status of said cell, the cell must comprise a chimeric transmembrane receptor whose intracellular domain represents or comprises the known reaction partner of the potential intracellular interaction, preferably binding partner. On the other hand, if the cell is used, e.g., in a screening setup for identifying (so far unknown) reaction partners it is conceivably not necessary to have chimeric transmembrane receptors being reactive towards the potential intracellular interaction, preferably binding partner, since only through screening the test agent's, i.e. the potential intracellular interaction, preferably binding partner's reaction capacity and specificity, a reaction pair relationship between a potential intracellular interaction, preferably binding partner and a chimeric transmembrane receptor may be established. Also envisaged is a combination of the above in the same cell, i.e. the presence of chimeric transmembrane receptors known to interact with one or more potential intracellular interaction, preferably binding partners, combined with transmembrane receptors whose reaction capacity to said one or more potential intracellular interaction, preferably binding partners is not known, i.e. is to be evaluated.

In other words, different “bait” domains are present in the cytoplasm as part of transmembrane receptors, whereas as “prey” a candidate reactant is used. Molecules that can be used as potential intracellular interaction, preferably binding partner may be molecules endogenously occurring in the cell or molecules not endogenously occurring in the cell. In this regard it is preferred that the potential intracellular interaction, preferably binding partner is heterologously expressed in the cell. Heterologous expression may be present in addition to endogenous expression. For example, molecules include but are not limited to peptides, polypeptides, lipids, nucleic acid molecules, small molecules, prodrugs, drugs, second messengers or metabolites. Preferably, the potential intracellular interaction, preferably binding partner is a peptide or a polypeptide. It is understood that in accordance with the method of the invention more than a single potential intracellular interaction, preferably binding partner molecule is present within the cell. Preferably, the cell comprises a multitude of potential intracellular interaction, preferably binding partners of a kind such as, e.g. at least (for each value) 100, 250, 500, 1000, 2000, 3000, 4000, 5000, 10000, 50000, 100000, 10000000 or at least 100000000 potential intracellular interaction, preferably binding partners of a kind. As is known in the art (Molecular Cell Biology. 4th edition. Lodish H, Berk A, Zipursky S L, et al. New York: W. H. Freeman; 2000. Section 1.2 The Molecules of Life), about 108 molecules of an abundant protein like actin is estimated to be present per cell.

The cell according to the invention can be any cell provided that it comprises i. and ii. In case a known cell comprises i., e.g. a chimeric transmembrane receptor, introduction of ii. (or nucleic acid(s) encoding ii.) will deliver a cell according to the invention. Cells suitable for said modification and to be used in accordance with the method of the invention can be derived from existing cells lines or obtained by various methods including, for example, obtaining tissue samples in order to establish a primary cell line. Methods to obtain samples from various tissues and methods to establish primary cell lines are well-known in the art (Jones G E, Wise C J., “Establishment, maintenance, and cloning of human dermal fibroblasts.” Methods Mol Biol. 1997; 75:13-21). Suitable cell lines may also be purchased from a number of suppliers such as, for example, the American tissue culture collection (ATCC), the German Collection of Microorganisms and Cell Cultures (DSMZ) or PromoCell GmbH. Such cells may be mammalian cells such as, e.g., human, primate, rodent or bovine cells. Said cells may be somatic cells or germline cells such as, e.g., fibroblasts, heaptocytes, splenocytes, lymphocytes, spermatogonial cells, embryonic stem cells. Said cells may have to be manipulated, preferably transformed, prior to their use in the method of the invention to allow expression of many different labelled and unlabelled proteins simultaneously, i.e. the chimeric transmembrane receptors and the potential intracellular interaction, or preferably binding partners. For example, bacterial artificial chromosomes harboring sequences encoding for said receptors and potential intracellular interaction, or preferably binding partners can be constructed. Also envisaged is the use of cells that have been manipulated to dedifferentiate from a unipotent or multipotent state into a pluripotent state being comparable to that of embryonic stem cells. Said cells are commonly referred to as induced pluripotent stem cells; means and methods to generate said cells are well-known in the art (see e.g., Takahashi et Yamanaka, Cell, (2006) 126:663-676; Wernig et al., Nature, (2007) 448:318-324).

The term “transmembrane receptor” designates a protein that is capable to span the plasma membrane of a cell. Naturally-occurring transmembrane receptors (i.e. the endogenous transmembrabe receptors of a cell) have in addition to a transmembrane domain in general an (i) extracellular binding domain having the ability to bind to a ligand and (ii) an intracellular domain having an activity (such as a kinase activity) that can be altered (either increased or decreased) upon ligand binding. There are two basic types of naturally-occurring transmembrane proteins. (i) Alpha-helical: These proteins are present in the inner membranes of bacterial cells or the plasma membrane of eukaryotes, and sometimes in the outer membranes of bacteria. This is the major category of transmembrane proteins. In humans, 27% of all proteins have been estimated to be alpha-helical membrane proteins. Beta-barrels. These proteins are so far found only in outer membranes of Gram-negative bacteria, cell wall of Gram-positive bacteria, and outer membranes of mitochondria and chloroplasts. All beta-barrel transmembrane proteins have simplest up-and-down topology, which may reflect their common evolutionary origin and similar folding mechanism. Another classification of naturally-occurring transmembrane proteins refers to the position of the N- and C-terminal domains. Types I, II, and III are single pass molecules, while type IV are multiple pass molecules. Type I transmembrane proteins are anchored to the lipid membrane with a stop-transfer anchor sequence and have their N-terminal domains targeted to the ER lumen during synthesis (and the extracellular space, if mature forms are located on plasmalemma). Type II and III are anchored with a signal-anchor sequence, with type II being targeted to the ER lumen with its C-terminal domain, while type III have their N-terminal domains targeted to the ER lumen. Type IV is subdivided into IV-A, with their N-terminal domains targeted to the cytosol and IV-B, with an N-terminal domain targeted to the lumen. The implications for the division in the four types are especially manifest at the time of translocation and ER-bound translation, when the protein has to be passed through the ER membrane in a direction dependent on the type (Harvey Lodish etc.; Molecular Cell Biology, Sixth edition, p. 546).

A “chimeric transmembrane receptor” (also designated herein artificial transmembrane receptor or bait presenting artificial receptor construct (bait-PARC)) as used herein defines a transmembrane receptor, wherein at least two (the two domains either being domains (a) and (b); domains (a) and (c), or domains (c) and (b)) and preferably all three of the domains (a), (b) and (c) are of different origin. “Of different origin” means that the source of domains (a), (b) and (c) or from where domains (a), (b) and (c) are derived is different. As such, the chimeric transmembrane receptor of the invention cannot be a naturally-occurring transmembrane receptor or cannot have an amino acid sequence being identical to a naturally-occurring transmembrane receptor. The chimeric transmembrane receptor is an artificially designed protein construct not existing in nature.

It is understood that in accordance with the method of the invention preferably more than a single copy of each distinct chimeric transmembrane receptor is present in the cell membrane. Preferably, the cell comprises a multitude of chimeric transmembrane receptors of a kind such as, e.g. at least (for each value) 100, 250, 500, 1000, 2000, 3000, 4000, 5000, 10000, 50000 or at least 100000 chimeric transmembrane receptors. Also preferred is that the amounts of the distinct chimeric transmembrane receptors are essentially equal in a cell to be used in accordance with the invention. Amounts that are considered to be essentially equal are amounts that differ by less than 1%, 2%, 3%, 4%, 5%, 10% or 20%. Nevertheless, it is also envisaged that the amounts vary by more than the recited percentages. Also preferred is that the cell comprises more than the at least two different kinds of chimeric transmembrane receptors such as, e.g., at least 3, 4, 5, 6, 7, 8, 9, 10, 15, 20, 30, 40, 50, 100 or at least 200 distinct chimeric transmembrane receptors.

Said chimeric transmembrane receptors are inter alia characterized in that the extracellular binding domain specifically interacts with an extracellular compound that is not bound by other chimeric transmembrane receptors to be used in accordance with the method of the invention. The purpose of the extracellular binding domain is to enable the recruitment of the transmembrane receptor based on extracellular compounds immobilized on a surface of (a) support(s). Preferably, said specific interaction is a direct interaction, i.e. a specific binding occurs between extracellular compound and an extracellular binding domain of a chimeric transmembrane receptor. Preferably, a chimeric transmembrane receptor does not interact with an extracellular compound that is also bound by transmembrane receptors endogenously occurring on the cell but not employed in the method of the invention. Specific interaction as used herein means that said chimerc transmembrane receptor exclusively, i.e. specifically, interacts with a (target) extracellular compound and may be described, for example, in terms of cross-reactivity. Preferably, and in the case of extracellular compounds being peptides or polypeptides “specifically binding” refers to chimeric transmembrane receptors that do not bind to a peptide or polypeptide with less than 100%, 99.999%, 99.99%, 99.9%, 99%, 98%, 95%, 90%, 85%, 80%, 75%, 70% or less than 65% identity (as calculated using methods known in the art) to the peptide or polypeptide involved in binding and to which specific binding must occur, i.e. the target extracellular compound. The chimeric transmembrane receptor may, however, also be described or specified in terms of its binding affinity between the extracellular binding domain of the transmembrane receptor and the extracellular compound. Preferred binding affinities include those with a dissociation constant or Kd less than 5×10-6M, 10-6M, 5×10-7M, 10-7M, 5×10-8M, 10-8M, 5×10-9M, 10-9M, 5×10-10M, 10-10M, 5×10-11M, 10-11M, 5×10-12M, 10-12M, 5×10-13M, 10-13M, 5×10-14M, 10-14M, 5×10-15M, and 10-15M. If the interaction is an indirect interaction, i.e. the extracellular binding domain binds to another molecule that is in turn bound by the extracellular compound, the above applies also with regard to the interaction of the extracellular binding domain to said another molecule and the interaction of said extracellular compound to said another molecule.

Specific binding occurs at a defined site of the target molecule and goes along with the formation of a network of several distinct and specific interactions. Specific binding may occur with hardly any change of the conformation of the molecules involved (“key-in-lock”), or it may involve conformational changes of one or both of the binding partners (“hand-in-glove” paradigm). Binding involves interaction between one or more moieties or functional groups of the chimeric transmembrane receptor and one or more moieties or functional groups of an extracellular compound, wherein said interaction may comprise one or more of charge-charge interactions; charge-dipole interactions; dipole-dipole interactions, wherein said dipoles may be permanent, induced or fluctuating; hydrogen bonds; and hydrophobic interactions. Hydrogen bonds and interactions involving a permanent dipole are of particular relevance in the sense that they confer specificity of binding by their directional character.

The integration of chimeric transmembrane receptors into the cell membrane of said cell used in the method of the invention which are not endogenously occurring in the cell can be achieved, e.g., by transfecting the cell with nucleic acid sequences encoding chimeric transmembrane receptors which upon expression are incorporated into the cell membrane via the cell's endogenous mechanisms or by fusing cell membranes of the cell with a membrane from another cell which carries chimeric transmembrane receptors to be used in the method of the invention.

The term “extracellular binding domain” refers to a protein domain being located outside the cell and which binds to a specific atom or molecule, such as calcium, DNA, polypeptide or protein. Upon binding, the binding domain may undergo a conformational change. Hence, the extracellular binding domain is the part of the receptor that sticks out of the membrane on the outside of the cell. Preferably, the extracellular binding domain of the invention does not interact with an extracellular compound that is also bound by transmembrane receptors endogenously occurring on a cell. The extracellular binding domain preferably specifically binds to a specific atom or molecule.

The term “polypeptide” is used herein interchangeably with the term “protein” and describes linear molecular chains of amino acids, including single chain proteins or their fragments, containing more than 30 amino acids. Polypeptides may further form oligomers consisting of at least two identical or different molecules. The corresponding higher order structures of such multimers are, correspondingly, termed homo- or heterodimers, homo- or heterotrimers etc. Homodimers, trimers etc. of fusion proteins giving rise or corresponding to enzymes also fall under the definition of the term “polypeptide”. Furthermore, peptidomimetics of such proteins/polypeptides where amino acid(s) and/or peptide bond(s) have been replaced by functional analogues are also encompassed by the invention. Such functional analogues include all known naturally occurring or synthetic amino acids other than the 20 gene-encoded amino acids, such as selenocysteine or ketone-functionalized amino acids. The terms “polypeptide” and “protein” also refer to naturally or synthetically modified polypeptides/proteins where the modification is effected e.g. by glycosylation, acetylation, phosphorylation and similar modifications which are well known in the art. The above applies mutatis mutandis also to the term “peptide” which as used herein describes a group of molecules consisting of up to 30 amino acids.

A “transmembrane domain” is in accordance with the invention any three-dimensional protein structure which is thermodynamically stable in a cell membrane and spans the cell membrane. This may be, for example, a single alpha helix, a stable complex of several transmembrane alpha helices, a transmembrane beta barrel, or a beta-helix of gramicidin A. Transmembrane helices are usually about 20 amino acids in length.

The term “intracellular domain” (or cytoplasmic domain) as used herein defines a domain which potentially interacts with the interior of a cell or a cellular organelle. In other words, the term “intracellular domain” in its broadest form embraces candidate intracellular domains. For example, the capability of a given potential intracellular interaction, preferably binding partner as candidate ligand to interact with a variety of different intracellular domains of different chimeric transmembrane receptors can be tested. In other words, different “bait” domains are present in the cytoplasm as part of transmembrane receptors, whereas as “prey” a candidate binding partner. Intracellular domains may be molecules endogenously occurring in a cell or molecules not endogenously occurring in a cell. For example, molecules include but are not limited to peptides, polypeptides, lipids, nucleic acid molecules, small molecules, prodrugs, drugs, second messengers or metabolites. Preferably, the potential intracellular interaction, preferably binding partner is a peptide, polypeptide or DNA. For example, the intracellular domain may form specific protein-protein-interactions or protein-DNA-interactions inside the cell. Alternatively, the intracellular domain may have enzymatic activity, such as a tyrosine kinase activity.

The term “lipid” is well known in the art and relates to predominantly lipophilic/hydrophobic molecules which may carry a polar headgroup. Lipids according to the invention include simple lipids such as hydrocarbons (triacontane, squalene, carotinoids), alcohols (wax alcohol, retinol, cholesterol, linear mono- or polyhydroxylated hydrocarbons, preferably with two to about 30 carbon atoms), ethers, fatty acids and esters such as mono-, di- and triacylgylcerols. Furthermore included are complex lipids such as lipoproteins, phospholipids and glycolipids. Phospholipids in turn comprise glycerophospholipids such as phosphatidic acid, lysophosphatidic acid, phosphatidylgylcerol, cardiolipin, lysobisphosphatidic acid, phosphatidylcholine, lysophosphatidylcholine, phosphatidylethanolamine, phosphatidylserine, phosphatidylinositol and phosphonolipids. Glycolipids include glycoglycerolipids such as mono- and digalactosyldiacylgylcerols and sulfoquinovosyldiacylgylcerol. Also included by the term “lipid” according to the present invention are sphingomyelin glycosphingolipds and ceramides.

The term “nucleic acid” or “nucleic acid compound”, in accordance with the present invention, includes DNA, such as cDNA or genomic DNA, and RNA. It is understood that the term “RNA” as used herein comprises all forms of RNA including mRNA. The term “nucleic acid molecule” is interchangeably used in accordance with the invention with the term “polynucleotide”.

Further included are nucleic acid mimicking molecules known in the art such as synthetic or semisynthetic derivatives of DNA or RNA and mixed polymers, both sense and antisense strands. They may contain additional non-natural or derivatized nucleotide bases, as will be readily appreciated by those skilled in the art. Nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA) and locked nucleic acid (LNA) (see, for example, Braasch and Corey, Chemistry & Biology 8, 1-7 (2001)). LNA is an RNA derivative in which the ribose ring is constrained by a methylene linkage between the 2′-oxygen and the 4′-carbon.

For the purposes of the present invention and as known in the art, a peptide nucleic acid (PNA) is a polyamide type of DNA analog. The monomeric units for the corresponding derivatives of adenine, guanine, thymine and cytosine are commercially available (for example from Perceptive Biosystems). PNA is a synthetic DNA-mimic with an amide backbone in place of the sugar-phosphate backbone of DNA or RNA. As a consequence, certain components of DNA, such as phosphorus, phosphorus oxides, or deoxyribose derivatives, are not present in PNAs. As disclosed by Nielsen et al., Science 254:1497 (1991); and Egholm et al., Nature 365:666 (1993), PNAs bind specifically and tightly to complementary DNA strands and are not degraded by nucleases. Furthermore, they are stable under acidic conditions and resistant to proteases (Demidov et al. (1994), Biochem. Pharmacol., 48, 1310-1313). Their electrostatically neutral backbone increases the binding strength to complementary DNA as compared to the stability of the corresponding DNA-DNA duplex (Wittung et al. (1994), Nature 368, 561-563; Ray and Norden (2000), Faseb J., 14, 1041-1060). In fact, PNA binds more strongly to DNA than DNA itself does.

PNA chimera according to the present invention are molecules comprising one or more PNA portions. The remainder of the chimeric molecule may comprise one or more DNA portions (PNA-DNA chimera) or one or more polypeptide portions (peptide-PNA chimera). Peptide-DNA chimera according to the invention are molecules comprising one or more polypeptide portions and one or more DNA portions. Molecules comprising PNA, peptide and DNA portions are envisaged as well. The length of a portion of a chimeric molecule may range from 1 to n−1 bases, equivalents thereof or amino acids, wherein “n” is the total number of bases, equivalents thereof and amino acids of the entire molecule.

The term “derivatives” in conjunction with the above described PNAs, PNA chimera and peptide-DNA chimera relates to molecules wherein these molecules comprise one or more further groups or substituents different from PNA, polypeptides and DNA. All groups or substituents known in the art and used for the synthesis of these molecules, such as protection groups, and/or for applications involving these molecules, such as labels and (cleavable) linkers are envisaged.

The term “small molecule” as used herein may describe, for example, a small organic molecule. Organic molecules relate or belong to the class of chemical compounds having a carbon basis, the carbon atoms linked together by carbon-carbon bonds. The original definition of the term organic related to the source of chemical compounds, with organic compounds being those carbon-containing compounds obtained from plant or animal or microbial sources, whereas inorganic compounds were obtained from mineral sources. Organic compounds can be natural or synthetic. Alternatively the compound may be an inorganic compound. Inorganic compounds are derived from mineral sources and include all compounds without carbon atoms (except carbon dioxide, carbon monoxide and carbonates). Preferably, the small molecule has a molecular weight of less than about 2000 amu, or less than about 1000 amu such as 500 amu, and even less than about 250 amu. The size of a small molecule can be determined by methods well-known in the art, e.g., mass spectrometry. Small molecules may be designed, for example, in silico based on the crystal structure of potential drug targets, where sites presumably responsible for the biological activity and involved in the regulation of expression of genes identified herein, can be identified and verified in in vivo assays such as in vivo HTS (high-throughput screening) assays. Small molecules can be part of libraries that are commercially available, for example from ChemBridge Corp., San Diego, USA.

A “prodrug” in accordance with the invention is a compound that is generally not biologically and/or pharmacologically active. After administration, the prodrug is activated, typically in vivo by enzymatic or hydrolytic cleavage and converted to a biologically and/or pharmacologically active compound which has the intended medical effect, i.e. is a drug that exhibits a biological and/or pharmacologic effect. Prodrugs are typically formed by chemical modification of biologically and/or pharmacologically active compounds. Conventional procedures for the selection and preparation of suitable prodrug derivatives are described, for example, in Design of Prodrugs, ed. H. Bundgaard, Elsevier, 1985. Accordingly, the term “drug” as used herein is a compound that when absorbed after administration alters the bodily function through its biological and/or pharmacologic activity. Preferably, the drug is a compound used or a candidate compound intended for use in the treatment, cure, prevention or diagnosis or used or intended to be used to otherwise enhance physical or mental well-being.

The term “second messengers” as used herein is known in the art and refers to molecules that relay signals from receptors on the cell surface to target molecules inside the cell, in the cytoplasma or nucleus. For example, second messengers are involved in the relay of the signals of hormones or growth factors and are involved in signal transduction cascades. Second messengers may be grouped in three basic groups: hydrophobic molecules (e.g., diacyglycerol, phosphatidylinositols), hydrophilic molecules (e.g., cAMP, cGMP, IP3, Ca2+) and gases (e.g., nictric oxide, carbon monoxide).

The term “metabolites” as used herein corresponds to its generally accepted meaning in the art, i.e. metabolites are intermediates and products of metabolism and may be grouped in primary (e.g., involved in growth, development and reproduction) and secondary metabolites.

In accordance with the method of the invention, said one or more potential intracellular interaction, preferably binding partners are labeled with a first label. Said first label is characterized in that it can be differentiated from a second label in cases where a second label is used in the method according to the invention (see below). It is understood that in certain experimental setups it may suffice to label different potential intracellular interaction, preferably binding partners with the same first label as long as a correlation of the detected signal to a chimeric transmembrane receptor is unambiguously possible as discussed further below in connection with the knowledge of the position or coordinate of an area comprising a specific extracellular compound on a support. In other embodiments, each different potential intracellular interaction, preferably binding partner is labelled with a different first label. In other embodiments, the first label of a given potential intracellular interaction, preferably binding partner may be a chromophore used as a probe in connection with a second chromophore (e.g., as second label) that is preferably attached to the intracellular domain of the cognate chimeric transmembrane receptor to take advantage of the principle of fluorescence resonance energy transfer (FRET; also known as Förster resonant energy transfer) that is well-known in the art as are suitable probes. Preferably, said chromophore used as first label is the acceptor chromophore that is excited when the second chromophore is in proximity (typically less than 10 nm) and whose (altered) signal can be detected. Using corresponding probes as first and second labels additionally provides the option to assess whether the reaction (being preferably a binding) between potential intracellular interaction, preferably binding partner and chimeric transmembrane receptor is direct or indirect on the basis of a comparison of fluorescent intensities in view of suitable control samples. To this end, modifications of FRET such as BRET (bioluminescence resonance energy transfer) are also envisaged. To the extent second label(s) are used in the method according to the invention, it is understood that said first label(s) can be differentiated from said second label(s). It is also understood that in certain experimental setups it may suffice to label different complexes with the same second label as long as a correlation of the detected signal to a chimeric transmembrane receptor is unambiguously possible in connection with the knowledge of the position or coordinate of an area comprising a specific extracellular compound on a support. The kind of label and method employed for labeling essentially depends on the nature of the potential intracellular interaction, preferably binding partner to be used in accordance with the method invention. In the case of peptides and polypeptides, the label is preferably a fluorescent protein or a fluorescent dye. Accordingly, in the first case said peptides or polypeptides may be labeled by creating a fusion protein comprising said fluorescent protein and said protein or polypeptide. Alternatively, e.g. in the case of a fluorescent dye, the label may be linked to said peptide or polypeptide. Preferably, the label is green fluorescent protein (GFP) or spectrally distinguishable variants such as, e.g., BFP, CFP, YFP, mRFP1, phytochrome derived far-red or near infrared fluorescent proteins such as e.g., IFPs (Shu et al. Science. 2009 May 8; 324(5928):804-7) or variants that exhibit a large Stokes shift, e.g. Keima. More preferred is that the first label is a bright and stable fluorescent protein such as, e.g., EGFP or mRFP derivatives such as mCherry. A label may also comprise more than one compound, e.g. two fluorophores which generate a combination color.

As defined in the main embodiment, in each support and its cognate transmembrane receptor the intracellular domain may be labelled with a label or a pair of labels which is capable to indicate reactions with one or more potential intracellular interaction, preferably binding partners of step a.ii. with the intracellular domain. Under this scenario FRET-based sensors are preferrably employed, in which two labels are incorporated into the intracellular domain of a chimeric transmembrane receptor. In that case, reactions of this intracellular domain with, or preferably binding of this intracellular domain to an intracellular interaction/binding partner—even if the later is not directly labeled by a first label—can induce a conformational change in the intracellular domain of a chimeric transmembrane receptor, which is then detected via changes in FRET efficiency. It thus has to be understood that if such FRET-based sensors are used in accordance with the invention, one or more different potential intracellular interaction, preferably binding partners may not be directly labelled, but instead intracellular reactions or interactions (such as binding) are indirectly detected by measuring a conformational change in the intracellular domain of a chimeric transmembrane receptor in which two labels are incorporated into the intracellular domain (one of which may be deemed the first label as defined in ii.a of the main embodiment) thereby constituting the discussed FRET-based sensor. Instead of a FRET-based sensor (or more precisely a donor-acceptor pair for measuring FRET), also a BRET-based sensor (bioluminescence resonance energy transfer) pair can be used. Also BRET-based sensor are well-known in the art (see, e.g. Xu et al (1998), PNAS, 96(1): 151-156).

The term “contacting” as used in connection with the method of the present invention means bringing the cell and the at least two different extracellular compounds into proximity such that the extracellular compounds can specifically interact with the extracellular binding domain of its cognate chimeric transmembrane receptor. Said contacting is performed in conditions suitable for allowing an interaction with said cell and at least two different extracellular compounds. Suitable conditions for contacting are, e.g., contacting in an aqueous solution, in a buffered solution. It is understood that such conditions and solutions, respectively, are suitable for cell integrity. Preferred embodiments make use of cell culture media. The explanations of specific interaction and specific binding as described herein above apply mutatis mutandis also to the interaction of the extracellular domains of said at least two chimeric transmembrane receptors and said at least two extracellular compounds. The specific interaction, preferably the specific binding of each extracellular compound to its cognate chimeric transmembrane receptor is mandatory for successfully working the method of the invention, whereas a specific interaction with the potential intracellular interaction, preferably binding partner to the chimeric transmembrane receptor may or may not be mandatory depending on the specific problem to be addressed as described herein above and below.

The “extracellular compound” according to the invention may be any molecule or complex of the same or different molecules that is capable of specifically interacting with the extracellular binding domain of a chimeric transmembrane receptor and, optionally, that can be labelled. The “at least two distinct compounds binding with the extracellular binding domains” must be different in that each compound is exclusively bound by one kind of chimeric transmembrane receptor of the cell of the invention. Such molecules include but are not limited to peptides, polypeptides including antibodies and fragments thereof, nucleic acid molecules, aptamers, oligosaccharides or synthetic protein-binding agents. Preferred extracellular compounds are nucleic acid molecules such as, e.g. DNA molecules; and polypeptides such as, e.g. antibodies or antibody fragments.

“Synthetic protein-binding agents” are small molecules that interact with proteins or combinations of such small molecules or fragments thereof, linked via a scaffold to create multivalent binders. Several examples are given in: Kodadek et al., Acc. Chem. Res. 37:711, such as for example a subpicomolar inhibitor of acetylcholine esterase (Lewis et al., Angew. Chem., Int. Ed. 41:1053) or a variant termed “Mixed Element Capture Agents” (MECAs), in which the scaffold is not a typical molecular linker, but instead a surface on which different non-competitive linkers are immobilized. Also included are Protein Surface Mimetics, which can be based on peptidic or non-peptidic structures, which differ from peptides or polypeptides in their more rigid structure due to additional stabilizing bonds (Cummings C G, Hamilton A D. Curr Opin Chem Biol. 14:341 and Hershberger S J Curr Top Med Chem. 7:928).

The term “nucleic acid molecules” has been defined herein above. Such molecules, when used as compound binding with the extracellular binding domain of said chimeric transmembrane receptor offer the advantage that they are both biologically stable and generally do not interact with endogenous cell surface receptors. Furthermore, a wide diversity of orthogonal pairs, i.e. pairs whose partners exclusively interact with each other, of chimeric transmembrane receptors and compound binding with the extracellular binding domain of said chimeric transmembrane receptor can be generated, e.g. when the compounds consist of or comprise zinc-finger DNA binding domains that can be permutated to guarantee specific interaction with a DNA molecule as extracellular compound. Also, nucleic acid molecules used as compounds can, of course, be conveniently labeled by incorporating or attaching, e.g., a radioactive, fluorescent, luminescent or other marker. Such markers are well known in the art. The labeling of said nucleic acid molecules can be effected by conventional methods.

The antibodies can be monoclonal antibodies, such as Fab, Fv or scFv fragments etc. Furthermore, antibodies or fragments thereof to the aforementioned polypeptides can be obtained by using methods which are described, e.g., in Harlow and Lane “Antibodies, A Laboratory Manual”, CSH Press, Cold Spring Harbor, 1988. The antibody used as compound specifically interact with the extracellular binding domain of the chimeric transmembrane receptor. The term “specifically interacts” or “specifically binds” as used in this context means that the antibody does not or essentially does not cross-react with a similar structure of another chimeric transmembrane receptor used in accordance with the invention. Preferably, the antibody or fragment thereof does not interact with an endogenous protein accessible on the cell surface. It is understood that the effect on normal cell behaviour is minimized by using such an antibody. Cross-reactivity of antibodies may be tested, for example, by assessing binding of said antibodies under conventional conditions to the epitope of interest as well as to a number of more or less (structurally and/or functionally) closely related epitopes. Only those antibodies that bind to the epitope of interest in its relevant context (e.g. a specific motif in the structure of a protein) but do not or not essentially bind to any other epitope are considered specific for the epitope of interest and thus to be antibodies that can be preferably used as a compound in accordance with this invention. Corresponding methods are described e.g. in Harlow and Lane, 1988 and 1999.

Aptamers are oligonucleic acid or peptide molecules that bind a specific target molecule. Aptamers are usually created by selecting them from a large random sequence pool, but natural aptamers also exist in riboswitches. Further, they can be combined with ribozymes to self-cleave in the presence of their target molecule. More specifically, aptamers can be classified as DNA or RNA aptamers or peptide aptamers. Whereas the former consist of (usually short) strands of oligonucleotides, the latter consist of a short variable peptide domain, attached at both ends to a protein scaffold. Nucleic acid aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Peptide aptamers consist of a variable peptide loop attached at both ends to a protein scaffold. This double structural constraint greatly increases the binding affinity of the peptide aptamer to levels comparable to an antibody's (nanomolar range). The variable loop length is typically comprised of 10 to 20 amino acids, and the scaffold may be any protein, which has good solubility properties. Currently, the bacterial protein Thioredoxin-A is the most used scaffold protein, the variable loop being inserted within the reducing active site, which is a -Cys-Gly-Pro-Cys- loop in the wild protein, the two cysteins lateral chains being able to form a disulfide bridge. Peptide aptamer selection can be made using different systems, but the most used is currently the yeast two-hybrid system. Aptamers offer the utility for biotechnological and therapeutic applications as they offer molecular recognition properties that rival those of the commonly used biomolecules, in particular antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Hence, a suitable extracellular compound is an aptamer specifically binding to a chimeric transmembrane receptor of the cell used in the method of the invention.

The term “oligosaccharides” is well-known in the art and refers to saccharide polymers containing a small number of component sugars such as, e.g., at least (for each value) 3, 4, 5, 6, 7, 8, 9, 10, 11, 12, 13, 14, or at least 15 monosaccharides. They may, e.g., be O- or N-linked to amino acid side chains of polypeptides or to lipid moieties.

The extracellular compounds are immobilized on a surface of a support. The term “support” as used in the present invention corresponds to its accepted meaning in the art. It provides a surface for the attachment of the at least two different extracellular compounds. Said surface according to the invention may be any surface. The surface may be a coating applied to the support or carrier, or the surface of the support or carrier itself may be used. Support or carrier materials commonly used in the art and comprising synthetic material, glass, plastic, gold, stainless steel, Teflon, nylon and silica are envisaged for the purpose of the present invention. Coatings according to the invention, if present, include poly-L-lysine- and amino-silane-coatings as well as epoxy- and aldehyde-activated surfaces. Preferably, the support is miniaturised, for example in the form of a chip, a disk, a bead or a microtiter plate. The support permits the simultaneous and, preferably, the parallel analysis of individual reactions (preferably interactions) and consequently multiple reactions (preferably multiple interactions) in a small amount of sample material, i.e. preferably only one cell or only a part of a cell. The choice of the support may depend on the method of detection that is employed and the specific arrangement of the detection device with regard to the support comprising the extracellular compounds bound to the cell's chimeric transmembrane receptors. For example, when fluorescent labels are used as first labels and the extracellular compounds are coated onto cover slips facing the cell and the signal emitted is visualized by microscopy the support must be translucent, i.e. allowing the penetration of the wavelength to be detected and the range of wavelengths emitted by the microscope's light bulb. In the case of objective-based TIRF detection of chimeric transmembrane receptor recruitment and reaction, preferably interaction, a material of suitable refractive index and thickness must be selected to allow formation of the evanescent wave. The skilled person in the filed is aware of materials that have a suitable refractive index and thickness. Preferably, in the case of supports that are not designed to be internalized, the support is compatible with fluorescence-based measurements such as, e.g. a glass support. The skilled person is in the position to select a suitable support based on the specific implementation of the method of the invention that he has chosen in order to determine whether a reaction, preferably an interaction occurs.

Means and methods for immobilization of said extracellular compounds on the surface of a support depends on the choice of the extracellular compound, the surface and/or the support and are known to the skilled person and/or can be devised or enhanced by routine experimental work. Immobilization may be achieved by semi- or fully automated methods such as, e.g., using a spotting roboter that may be configured to apply different extracellular compounds at a desired position, in a specific shape and/or size on the same or different supports. Immobilization may be effected, e.g., in the case of nucleic acid oligonucleotides by nano- or photolithography or by establishing non-covalent interactions using, e.g., biotin/streptavidin and/or related molecules such as neutravidin. Preferably, immobilization is effected via establishing covalent interactions, for example via coating of the support with silanes and subsequent chemical modifications and subsequent attachment of extracellular compounds.

The at least two different extracellular compounds may be immobilized on the same support or on different supports. When immobilized on the same support, the different kinds of extracellular compounds are immobilized in different areas of said support, i.e. they are not mixed but separated. The different areas may be immediately adjacent to each other or may be separated by an area not comprising an extracellular compound. An area comprising a specific extracellular compound may be present on said same support once or multiple times such as, e.g. at least (for each value) 2, 3, 4, 5, 10, 20, 30, 40, 50, 100, 200, 300, 400, 500, 1000, 5000, 10000, 50000 or at least 100000 times. It is preferred that each area is assigned a defined coordinate on the support, which is known to the person executing the method of the invention, in order to permit evaluation of the results, e.g., by adding fiduciary marks (being spots of special size/shape or out-of-register positions) on the support to assist in the definition of coordinates for areas containing distinct extracellular compounds. For example, when executing the method of the invention using only a first label and the first label is the same for two different potential intracellular interaction, preferably binding partners, it is essential to know the coordinates of the areas comprising a specific extracellular compound in order to properly interpret a first signal generated by said first label in a given area of said support, said signal being indicative of a reaction (being preferably an interaction). Hence, detecting the specific presence of a label in a given area of a support comprising a distinct extracellular compound indicates a reaction between a specific chimeric transmembrane receptor (binding to the respective extracellular compound) and a potential intracellular interaction, preferably binding partner. In other terms, the position on the support is used to decode the identity of the chimeric transmembrane receptor on which a reaction with a potential intracellular interaction preferable binding partner occurs.

In case second label(s) are used that are different for each of said at least two different extracellular compounds bound to their cognate chimeric transmembrane receptors, the knowledge of said coordinates is not mandatory for determining a reaction (being preferably an interaction) since instead said co-localization of said first and second signals provides the information necessary for determining a reaction (being preferably an interaction). In the case of known reaction, preferably interaction partners (being the potential intracellular interaction, preferably binding partner and chimeric transmembrane receptor) and if different, first labels are used for more than one potential intracellular interaction, preferably binding partner, the second label, the size or the shape may be the same while the coordinates of the areas comprising said at least two different extracellular compounds bound to said at least two chimeric transmembrane receptors must be known. This applies also to embodiments when screening for potential interaction, preferably binding partners.

The same effect, i.e. creating multiple areas, each comprising only one kind of extracellular compound, can be achieved by immobilizing the at least two different extracellular compounds on at least two different supports. For example, this may be implemented by beads, each bead carrying one or more copies of a given extracellular compound. The conditions with regard to labelling, size, shape and/or position apply also to this aspect of the method of the invention. The different supports each comprising one kind of said at least two different extracellular compounds must be in such a position respective to each other that they can be simultaneously covered by a single cell. Preferably, the cell is contacted with more than a single support of one kind of said at least two different extracellular compounds such as, e.g., at least (for each value) 2, 3, 4, 5, 10, 20, 30, 40, 50, 100, 200, 300, 400, 500, 1000, 5000 or at least 10000 depending on label, shape and/or size used.

Different supports may allow a modular approach when rearranging and/or exchanging extracellular compounds in order to generate a surface to which the cell can be contacted. It is understood in accordance with the present invention that combining different means for identifying the areas comprising extracellular compound bound to a chimeric transmembrane receptor such as, e.g., combining shape and second label, provides the advantage of increasing the combinatorial variety, thus allowing to determine the reaction, preferably interaction of more different potential intracellular interaction, preferably binding partners and chimeric transmembrane receptors.

A second label in accordance with the invention may be any label that can be differentiated from said (different) first label(s) and can be attached to an extracellular compound, a cognate chimeric transmembrane receptor and/or to the area of the support to which the extracellular compound has been immobilized. As described herein above, a second label may be necessary to allow assessment of co-localization of potential intracellular interaction, preferably binding partner and the complex of extracellular compound bound to its cognate chimeric transmembrane receptor. Preferably, the first and second labels are labels that can be detected using the same detection means. More preferred is that first and/or second labels are luminescent labels such as fluorescent and/or phosphorescent labels, such as e.g. fluorescent proteins (GFP, EGFP, YFP, CFP, RFP, IFP, Kaima), Alexa Fluor® dyes, AMCA®, BODIPY® 630/650, BODIPY® 650/665, BODIPY®-FL, BODIPY®-R6G, BODIPY®-TMR, BODIPY®-TRX, Cascade Blue®; CyDyes™, including but not limited to Cy2™, Cy3™, Cy5™, Cy7™; DNA intercalating dyes, 6-FAM™, Fluorescein, HEX™, 6-JOE, Oregon Green® 488, Oregon Green® 500, Oregon Green® 514, Pacific Blue™, REG; phycobilliproteins including but not limited to phycoerythrin, allophycocyanin, Rhodamin Green™ Rhodamin Red™, ROX™, TAMRA™, TET™, Tetramethylrhodamine, Texas Red®, Atto dyes, IRDye 680LT, IRDye 800CW and quantum dots. Even more preferred is that the first label is EGFP or mTurquoise and the second label is Atto-740 (www.atto-tec.com; ATTO-TEC GmbH, Germany), Alexa Fluor 750 or IRDye 800CW (www.licor.com; LI-COR Biotechnology, USA). The person skilled in the art is in the position to extend the number of first and second labels to be used in accordance with the method of the invention by fluorescent colour-coding of said extra- and potential intracellular interaction, preferably binding partners, the chimeric transmembrane receptors and/or the areas on the support to which said extracellular compounds are bound. Since infra-red fluorescent dyes and suitable excitation and detection devices are commonly available a multitude of distinct fluorescent colour combinations can be realized by using defined fluorescence intensities and ratios in mixtures of two different fluorescent dyes such as, e.g., the two near-infrared dyes ATTO 647 and ATTO 740.

As outlined herein above, the determinants shape and size of areas consisting of or comprising different extracellular compounds may in combination with the knowledge of their positions on a support or in combination with (a) signal(s) generated by a second label enable assessment of the co-localization of the complex of extracellular compound (immobilized on a support) bound to a cognate chimeric transmembrane receptor and a potential intracellular interaction, preferably binding partner. Preferably, at least the position of an area comprising one kind of an extracellular compound on a given support is known to the person executing the method of the invention.

The skilled person is in the position to select the kind and combination of first label with second label, shape, size and/or coordinate on the support so that this person can detect the co-localization of a chimeric transmembrane receptor being bound to an extracellular compound and a potential intracellular interaction, preferably binding partner and that this person can correlate said co-localization to a specific kind of chimeric transmembrane receptor and potential intracellular interaction, preferably binding partner.

The term “detecting” is used in accordance with its well-known meaning and refers to the process of recording the signals generated by the first and, if applicable, second label(s) and/or shape and/or size. The detection step must enable the person executing the method of the invention to assess whether a co-localization of the potential intracellular interaction, preferably binding partner and the binary complex of extracellular compound and cognate chimeric transmembrane receptor takes place. In case the position of the at least two different extracellular compounds on a support is known, only the first label(s) of the potential intracellular interaction, preferably binding partner(s) must be detected. Means and methods for detection depend on the specific setup, i.e. for example choice of support (material, same or different supports) and/or choice of first and second label(s) and can be chosen and implemented by the skilled person without further ado. Detection may be semi- or fully automated and include, e.g., signal analysis with respect to rotation of the image with respect to scan direction and support and/or extracellular compound comprising area size. The “image” in accordance with the present invention refers to the copy of, e.g., the fluorescent signals generated and detected with fluorescent microscopy and created, e.g., with a CCD-camera. In the case that detection does not occur continuously, i.e. intermittently, it is preferred that signal measurements are performed at least twice, more preferred at least (for each value) 2 times, 3, 4, 5 or 6 times for a given point in time, i.e. a reading point, in order to calculate the median as well as standard deviation. When using (a) fluorescent label(s) one can, e.g. use a fluorescence microscope. When using fluorescent labels both on the chimeric transmembrane receptor and on the potential intracellular interaction, preferably binding partner, advanced fluorescence detection methods, such as FRET or “fluorescence lifetime imaging microscopy” (FLIM), can provide information, whether the reaction (being preferably an interaction) between the chimeric transmembrane receptor and the potential intracellular interaction, preferably binding partner is direct or indirect. A preferred form of a fluorescent microscope to be used in conjunction with a glass support and one or more fluorescent labels is called “total internal reflection fluorescence microscope” (TIRFM). The skilled person is familiar with the setup of a corresponding device and how to use in accordance with the method of the invention. TIRF microscopy enables the specific detection and resolution of fluorescent labels that are bound to the cell's surface without being overwhelmed by background fluorescent signals of fluorescent labels in the surroundings, in particular in the cytosol of the cell. This is achieved by selective illumination and excitation of fluorescent labels in a restricted region of the cell, i.e. near the cell membrane bound to extracellular compounds.

As outlined above, after the detection of said first label and/or said first label and second label, shape and/or size, the person executing the method of the invention can assess whether a co-localization of the potential intracellular interaction, preferably binding partner and the complex of extracellular compound and cognate chimeric transmembrane receptor has taken place. When said at least two different extracellular compounds are immobilized on the same support, one can assess said co-localization on the basis of detection of said first label(s) and the knowledge of the position of an area comprising the cognate extracellular compound of one of the at least two chimeric transmembrane receptors. The same also applies with regard to said at least two different extracellular compounds on different supports when the supports can be distinguished, e.g. by their size, shape. Alternatively, the different supports comprising a given extracellular compound and/or the cognate chimeric transmembrane receptors are labeled with second labels, thus allowing an unambiguous assessment of co-localization of the signal of said first and said second label(s). Preferably, the assessment includes comparison of the signals detected to suitable positive and/or negative controls on the basis of which the detected signal(s) in step c. can be readily assessed. This may be important in experimental setups that result in a high level of unspecific background signals necessitating the comparison of a signal detected in areas on the support known not to comprise immobilized extracellular compound with areas known to comprise said extracellular compound. This subtractive approach in the assessment of detected signals is known in the art to yield reliable results, in particular if a signal indicating a reaction, preferably a binding is only marginally higher than an unspecific background signal.

In comparison to suitable negative and/or positive controls one can also relatively and absolutely quantify the reactions (being preferably interactions) taking place. In absolute quantification no known standards or controls are needed. The reaction (being preferably an interaction) can be directly quantified. As well-known in the art, absolute quantification may rely on a predertimed standard curve. In relative quantification the reaction is quantified relative to a reference reaction (such as known control interaction). Means and methods for absolute and relative quantification are known in the art and discussed in greater detail herein below. Also in the absence of controls, one can relatively quantify the interactions taking place when comparing e.g. fluorescence intensities of the signal(s) detected at different measurement points, different points being different locations in space and/or time.

The above provided definitions for example for peptides, polypeptides or nucleic acid molecules apply mutatis mutandis to other sections and embodiments herein below if not expressly stated otherwise.

In contrast to the limitations of previous approaches as outlined herein above, the present invention opens the way for simultaneous, even time resolved measurement of multiple protein reactions, in particular protein interactions in a single, individual cell. This unique feature of this invention enables extraction of detailed information about the dynamics of protein network states of an individual living cell. Furthermore, in contrast to previous studies, the approach extends beyond measuring naturally occurring reactions, in particular interactions between a wild-type receptor and a known reaction, in particular interaction partner. Here, multiplex biosensors are constructed, i.e. cell(s) to be used in accordance with the method of the invention, which can be tailored to contain any potential combination of artificial receptors, each containing distinct arbitrary intracellular domains (“bait”) and potential intracellular interaction, preferably binding partners (“prey”) of choice.

This principle also permits sensing the composition of the extracellular environment via readout of the live cell multiplex biosensor, i.e. the cell provided for use in the method of the invention as described herein above. This can be applied in biomedical research, clinical assay development or environmental monitoring of trace compounds that can elicit natural or engineered reactions in living cells. In particular, the simultaneous measurement of multiple protein reactions, in particular multiple protein interactions and protein reactions in an individual cell enables discrimination of multiple network states, which will allow sensitive and robust analysis readouts. Finally, by distinguishing different network states via multiplexed measurements, multiple components can be distinguished in complex extracellular assay mixtures.

For example, measurements of single or multiple peptide or polypeptide interactions or peptide or polypeptide reactions in an individual cell can be performed. Protein interactions play a pivotal role in cellular regulation both in physiological as well as pathophysiological conditions. Dynamic changes in protein interactions are indicative of their activity state and can thus be used to quantify biological processes at a level of molecular detail. While the measurement of an individual protein interaction or protein activity can be highly informative, many fundamental cellular processes, such as the determination of cell growth vs. cell shape changes are encoded by combinations of multiple dynamic activities. The described invention is enabling simultaneous measurements of multiple activities. The general implementation of this invention and the scalable concept of the cognate chimeric transmembrane receptor interacting with specific extracellular compounds based, e.g., on single-chain antibody fragments or zinc-finger DNA interactions offers a way to generate live cell multiplex biosensors to follow many protein interactions at the same time in an individual cell. Exemplarily, two concepts are described in the following.

Multiplex sensors for known, orthogonal protein reactions, preferably interaction pairs can be generated. The immobilized “bait” proteins, i.e. the intracellular domain of the chimeric transmembrane receptor, that are linked to said transmembrane receptors are readily distinguished, e.g., via spectral properties of functionalized beads or their relative positioning on a modified surface of support. The intracellular “prey”, i.e. the potential intracellular interaction, preferably binding partner, on the other hand can then be identified via, e.g., its fluorescent properties generated by a suitable first label. If the measurements are limited to only bimolecular and orthogonal reactions, preferably interactions (e.g. no direct cross talk—no direct cross modulation between the interaction pairs), multiple intracellular “prey” proteins can be labeled with the same fluorescent protein or dye, as only one intracellular “prey” protein would be able to interact with it's “bait” on the specific cognate chimeric transmembrane receptor. Examples of this type of multiplex biosensor could be composed of chimeric transmembrane receptors, which use, e.g., the GTPases Ras, RhoA and cdc42 as “bait”, which interact—in an activity dependent manner—specifically only with their cognate interaction domains (their “prey”) derived from Raf-kinase, rhotekin and N-Wasp, respectively. Such multiplex biosensors of orthogonal activity detection pairs can yield highly detailed information on the dynamics of interrelated signal activities in individual cells. For example, by analyzing the combinatorial dynamics of such activities, in combination with temporal perturbations, the dynamic interplay between individual components can be analyzed. The analysis of such interplay is not limited to the analysis of temporal dynamics: By using artificial receptors on mobile beads as sensors, or by generating repetitive arrays of few selected activities, the spatial distribution of activities can also be mapped, even within individual cells.

The method of the invention can also be used to identify new, i.e. unknown protein reactions, preferably interactions. For example, a single protein of interest can be linked to a fluorescent protein as the potential intracellular interaction, preferably binding partner (“prey”) and then be probed against a panel of immobilized “bait” candidate interaction, preferably binding partners, i.e. said intracellular domains of said at least two chimeric transmembrane receptors. In comparison to established techniques, such as yeast two-hybrid or mass spectrometry approaches, this invention allows the identification of novel protein reactions, preferably interactions in intact living cells in the natural context of the protein of interest. While the mass spectrometry approaches can address protein interactions in their natural context, the method can only be applied to cell extracts and therefore requires the destruction of the cells. On the other hand, the yeast two-hybrid system allows the identification of protein interactions in living cells, but it's biological context is limited to the nucleoplasm of yeast cells and therefore not a natural context for most applications.

The main advantage of the described invention in the context of identifying novel protein interactions is, however, the ability to dynamically manipulate the cellular context during the experiment. For example, a protein interaction might only be relevant during a particular dynamic state of the cell after hormonal cell stimulation or during a particular stage of the mitotic cycle, and therefore might be detectable only in a small subpopulation of cells. In cell extracts, an interaction that takes place only in a small subpopulation of cells might be masked by experimental noise. As this invention can be applied to the identification of new protein interactions in individual, living, intact cells, such dynamic, transient interactions, which might be elusive in other, standard methods, can be accessible via this invention.

The invention also enables the analysis of the cytoplasmic state of individual cells. The behaviour of individual cells is usually not defined by a single biological activity, but instead directed by a combinatorial set of activities, here denoted as a cytoplasmic state (Niethammer et al., 2007). Due to cell-to-cell variance, combinations of such activities can be very different between individual cells. In an analysis of the whole population, such differences will average out quickly, leading to a generalized readout of signals that is not representative of the original activity combinations of the original individual cells. Via the ability to study multiple protein interactions in individual living cells, this invention offers a way to determine the cytoplasmic state of individual cells. As the simplest example, one or more central signal molecules that form central nodes in interaction networks can be linked to fluorescent proteins and serve as potential intracellular interaction, preferably binding partners. Their interactions with many known interaction partners, which are differentially regulated by cellular signal network activities, can then be determined simultaneously by the method of the invention to determine the cytoplasmic state of this cellular signal network.

Our current knowledge about cytoplasmic states is very limited, as the few known examples required decades of laborious work to identify the individual interactions, their causal dependencies and their biological meaning. This invention can speed up this process by orders of magnitude, as it allows—for the first time—a straightforward and direct measurement of the real-time dynamics of cytoplasmic states. Direct correlation of cell behaviour—in unstimulated or stimulated conditions—with the measurements of cytoplasmic states defined by key regulatory signaling node interaction maps, will allow the rapid identification of behaviour specific cytoplasmic states of individual cells.

As a consequence of the invention, the development of cell-based sensors for clinical and environmental applications can be achieved. The knowledge that can be derived from measuring multiple protein interactions described herein above—and especially the correlation of cell behaviour with quantifiable cytoplasmic states, will allow the development of cell based sensors for compounds that induce changes in the cytoplasmic state. Medically relevant cytoplasmic states include for example apoptosis, necrosis, proliferation, transformation, senescence, differentiation, cell growth, cell shrinkage, etc. Detection devices that are based on such sensors include, but are not limited to: analysis of growth factors in medical samples or analysis of toxic test compounds. Furthermore, cells can be genetically engineered to express additional receptors for artificial compounds, such as controlled substances or explosives. If such receptors are linked to robust and sensitive cellular signal pathways, which induce a change in the cell's cytoplasmic state, and if this is combined with robust measurement of an altered cytoplasmic state via this invention, more robust and more sensitive detection devices for such substances can be generated.

Naturally-occurring transmembrane receptors have been used in the art to study protein interactions, for example, in WO 2008/080441. However the extracellular and the intracellular binding specificity as well as the cell signaling capability of naturally-occurring transmembrane receptors are predetermined. Furthermore, only a limited number of naturally-occurring transmembrane receptors exists. Most importantly, naturally-occurring transmembrane receptors and their ligands can perturb cellular function and thereby interfere with measurements of interest. By contrast and as also discussed above, the chimeric transmembrane receptor of the invention and the extracellular compound that interacts with this chimeric receptor by themselves are designed to minimally perturb cellular function. Their extracellular binding domain and/or an intracellular domain can then be selected as to have virtually any desired binding specificity and/or biological function. Furthermore, candidate intracellular interaction domains may be selected in order to elucidate potential intracellular reactions, preferably binding with the selected potential intracellular binding partner. The chimeric transmembrane receptors of the invention thus advantageously allow tailored multiplex analysis of various reactions, preferably multiple interactions within a single cell.

In accordance with a preferred embodiment of the method of the invention, the reaction is an interaction between a chimeric transmembrane receptor and an intracellular interaction partner or a protein conformational change of the chimeric transmembrane receptor and/or the intracellular interaction partner.

An “interaction” as used in accordance with the invention is either a direct physical interaction, also referred to as “binding”, or an indirect interaction mediated by other constituents that may or may not be endogenous components of the cell. As defined in the main embodiment, said reaction, preferably binding occurs within said cell. In other words, the reaction, preferably binding to be determined, occurs or may occur between said potential intracellular interaction, preferably binding partner and the intracellular domain of said receptor.

The term “determining whether an interaction occurs” has the established meaning in the art and extends to determining presence or absence of a given interaction, detecting whether a—possibly previously unknown—interaction occurs, quantifying interactions, wherein said interactions may include known as well as previously unknown interactions. The method according to the invention also extends to observing an interaction, wherein said observing may also include observing or monitoring over time and/or at more than one location, preferably locations within the cytoplasm or at the inner surface of the plasma membrane within a given cell. Such quantifying as well as monitoring in space and/or over time is the subject of preferred embodiments discussed further below.

The reaction to be determined by the method of the invention is preferably a protein reaction. The term “protein reaction” means that a chimeric transmembrane receptor changes its structure in response to changes in its environment, i.e. in response to a change within the cell. A “protein reaction” may be induced by many factors, such as a change in temperature, pH, voltage, ion concentration, phosphorylation, or the binding of a ligand. One type of protein reaction is a “conformational change”. If the conformational change alters the binding affinity of the chimeric transmembrane receptor to an intracellular binding partner, the change in the interaction strength may be determined as described above. The protein reaction of the chimeric transmembrane receptor may also include proteolytic cleavage. Means and methods for determining a protein reaction, and hence likewise protein reactions occurring on a chimeric transmembrane receptors are known in the art and described in greater detail herein below. For example, a FRET-based sensor is a highly suitable tool for detecting either conformational changes in a protein (when the distance between donor and acceptor changes), or proteolytic cleavage (when the donor and acceptor pair are separated after the cleavage of a substrate sequence) (see Neefjes & Dantuma (2004), Nature Reviews Drug Discovery 3, 58-69 for review). As defined herein above, a FRET-based sensor has to be associated with the intracellular domain.

According to a more preferred embodiment of the method of the invention, the (protein) reaction is selected from the group consisting of phosphorylation, glycosylation, lipidation (such as myristoylation, palmitoylation, prenylation), proteolytic cleavage, acetylation, disulfide bond formation, alkylation (such as methylation), ubiquitination, SUMOylation, oxidation, nitrosylation, nucleotide addition (such as ADP-ribosylation), adenylylation, arginylation, racemization of proline and the corresponding reverse reactions of the before listed reactions.

Phosphorylation, glycosylation, lipidation, proteolytic cleavage, acetylation, disulfide bond formation, alkylation (such as methylation), ubiquitination, SUMOylation, oxidation, nitrosylation, nucleotide addition (such as ADP-ribosylation), adenylylation, arginylation, racemization of proline and the corresponding reverse reactions of the before listed reactions are non-limiting examples of structural changes, which may be detected in accordance with the present invention, for example by detecting a conformational change triggered by such a structural change.

Phosphorylation is the addition of a phosphate group (PO43−) to a protein or other organic molecule. Reversible phosphorylation of proteins is an important regulatory mechanism that occurs in both prokaryotic and eukaryotic organisms. Kinases phosphorylate proteins and phosphatases dephosphorylate proteins. Hence, it is preferred that a potential intracellular interaction partner is a kinase or phosphate in case phosphorylation or desphosphorylation, respectively, is determined in accordance with the invention. For example, a FRET-based sensor associated with the intracellular domain of the chimeric transmembrane receptor may be used for determining whether de-/posphorylation occurs. In case such FRET-based sensor is used the potential intracellular interaction partner does not have to carry a label. FRET-based sensors for detecting phosphorylation are known in the art, for example, from Keese at al. (2005) Journal of Biological Chemistry, 280:27826-27831.; Nagai et al., Nat Biotechnol 2000 18:313-6.; Komatsu et al., Mol Biol Cell. 2011 22:4647-56.

Glycosylation is in accordance with the invention a reaction in which a carbohydrate, i.e. a glycosyl donor is attached to a hydroxyl or other functional group of the chimeric transmembrane receptor. In general, five classes of glycans may be produced, namely (i) N-linked glycans attached to a nitrogen of asparagine or arginine side-chains. N-linked glycosylation requires participation of a special lipid called dolichol phosphate; (ii) O-linked glycans attached to the hydroxy oxygen of serine, threonine, tyrosine, hydroxylysine, or hydroxyproline side-chains, or to oxygens on lipids such as ceramide; (iii) phospho-glycans linked through the phosphate of a phospho-serine; (iv) C-linked glycans, a rare form of glycosylation where a sugar is added to a carbon on a tryptophan side-chain; and (v) glypiation, which is the addition of a GPI anchor that links proteins to lipids through glycan linkages. Glycosylation is an enzymatic process within a cell. Glycosylation is a non-templated process (unlike DNA transcription or protein translation); instead, the cell relies on segregating enzymes into different cellular compartments (e.g., endoplasmic reticulum, cisternae in Golgi apparatus). Therefore, glycosylation is a site-specific enzymatic modification. The majority of glycosylation reactions occurs naturally in the extracellular compartment, however, glycosylation also occurs in the cytoplasm and in the nucleus. It is preferred that a potential intracellular interaction partner is a de-/glycosylation enzyme in case glycosylation or de glycosylation, respectively, is determined in accordance with the invention. For example, a FRET-based sensor associated with the intracellular domain of the chimeric transmembrane receptor may be used for determining whether de-/glycosylation occurs. In case such FRET-based sensor is used, the potential intracellular interaction partner does not have to carry a label. FRET-based sensors for detecting glycosylation are known in the art, for example, from Haga et al. (2012) Nature Communications 3, Article number: 907.

Lipidation as used herein is the covalent binding of a lipid group to a peptide chain of the chimeric transmembare receptor. Lipidation, can affect the activity of the protein and/or alter its subcellular location. For instance, palmitoylation, myristoylation or prenylation of cytoplasmic proteins can promote their association with the inner face of the plasma membrane, while the addition of a GPI-anchor may serve to anchor extracellular proteins to the outer face of the plasma membrane. In contrast to prenylation and myristoylation, palmitoylation is usually reversible (because the bond between palmitic acid and protein is often a thioester bond). Palmitoylation is an enzymatic process. The reverse reaction, depalmitoylation, is catalysed by palmitoyl protein thioesterases. Protein prenylation involves the transfer of either a farnesyl or a geranylgeranyl moiety to C-terminal cysteine(s) of the target protein. In cells, prenylation is catalysed by farnesyl and geranylgeranyl transferases. Myristoylation is catalyzed by the enzyme N-myristoyltransferase (NMT), and occurs most commonly on glycine residues exposed during co-translational N-terminal methionine removal. It is preferred that an intracellular interaction partner is a de-/lipidation enzyme in case lipidation or delipidation, respectively, is determined in accordance with the invention. For example, a FRET-based sensor associated with the intracellular domain of the chimeric transmembrane receptor may be used for determining whether de-/lipidation occurs. In case such FRET-based sensor is used, the potential intracellular interaction partner does not have to carry a label. FRET-based sensors for detecting lipidation are not commonly available, but can in principle be build by combining a substrate sequence (e.g., C-terminus of Cdc42), a specific lipid binding domain (e.g. RhoGDI; Hoffman et al., Cell. 2000 100:345-56) with a donor and acceptor fluorophore. The general guiding principle how such a sensor should be designed are known in the art (Miyawaki Annu Rev Biochem. 2011 80:357-73; Dehmelt and Bastiaens Nat Rev Mol Cell Biol. 2010 11:440-52).

Proteolytic cleavage or proteolysis is the breakdown of proteins into smaller polypeptides or amino acids. This generally occurs by the hydrolysis of the peptide bond, and is preferably achieved by cellular enzymes called proteases. It is preferred that a potential intracellular interaction partner is a protease in case proteolysis is determined in accordance with the invention. For example, a FRET-based sensor associated with the intracellular domain of the chimeric transmembrane receptor may be used for determining whether proteolysis occurs. In case such FRET-based sensor is used, the potential intracellular interaction partner does not have to carry a label. FRET-based sensors for detecting proteolysis are known in the art, for example, from Neefjes & Dantuma (2004), Nature Reviews Drug Discovery 3, 58-69.; Harpur et al., Nat Biotechnol. 2001 19:167-9.

Acetylation as sued herein is the introduction of an acetyl functional group into the chimeric transmembare receptor, while deacetylation is the removal of the acetyl group. Acetylation occurs as a co-translational and post-translational modification of proteins. Acetylation and deacetylation are enzymatic reactions. Proteins are typically acetylated on lysine residues and this reaction relies on acetyl-coenzyme A as the acetyl group donor. It is preferred that a potential intracellular interaction partner is a de-/acetylation enzyme in case acetylation or deactylation, respectively, is determined in accordance with the invention. For example, a FRET-based sensor associated with the intracellular domain of the chimeric transmembrane receptor may be used for determining whether de-/acetylation occurs. In case such FRET-based sensor is used, the potential intracellular interaction partner does not have to carry a label. FRET-based sensors for detecting acetylation are known in the art, for example, from Ito et al., Chem Biol. 2011 18:495-507; Sasaki et al., Bioorganic & Medicinal Chemistry 20:1887-1892.

A disulfide bond (SS-bond) is a covalent bond, usually derived by the coupling of two thiol groups. Disulfide bonds play an important role in the folding and stability of some proteins, usually proteins secreted to the extracellular medium. Disulfide bonds in proteins are formed between the thiol groups of cysteine residues. The formation of disulfide bonds is catalyzed by enzymes, such as protein disulfide isomerases and thiol-disulfide oxidoreductases. It is preferred that a potential intracellular interaction partner is an enzyme involved in disulfide bond formation in case disulfide bond formation is determined in accordance with the invention. For example, a FRET-based sensor associated with the intracellular domain of the chimeric transmembrane receptor may be used for determining whether disulfide bond formation occurs. In case such FRET-based sensor is used, the potential intracellular interaction partner does not have to carry a label. FRET-based sensors for detecting disulphide bond formation are known in the art, for example, from Yano et al., Mol Cell Biol. 2010 30:3758-66.; Oku et al., FEBS Lett. 2013 587:793-8.

Alkylation (such as methylation) is in accordance with the invention the introduction of an alkly (e.g. methyl) group into the the chimeric transmembare receptor. Methylation is the most common type of alkylation, being associated with the transfer of a methyl group. Methylation in nature is typically effected methyltransferases. In particular, histones are known to be methylated by specialized histone methyltransferases. It is preferred that a potential intracellular interaction partner is an enzyme involved in methylation in case methylation is determined in accordance with the invention. For example, a FRET-based sensor associated with the intracellular domain of the chimeric transmembrane receptor may be used for determining whether methylation occurs. In case such FRET-based sensor is used, the potential intracellular interaction partner does not have to carry a label. FRET-based sensors for detecting methylation are known in the art, for example, from Lin et al., J. Am. Chem. Soc., 126 (2004), p. 5982; Sasaki et al., Bioorganic & Medicinal Chemistry 20:1887-1892.

Ubiquitin is a small regulatory protein that has been found in almost all tissues (ubiquitously) of eukaryotic organisms. It directs proteins to compartments in the cell, including the proteasome which destroys and recycles proteins. Ubiquitination as used herein is the introduction of unbiquitin into the the chimeric transmembarane receptor. In more detail, Ubiquitination is an enzymatic, protein post-translational modification (PTM) process in which the carboxylic acid of the terminal glycine from the di-glycine motif in the activated ubiquitin forms an amide bond to the epsilon amine of the lysine in the modified protein. Ubiquitin is activated in a two-step reaction by an E1 ubiquitin-activating enzyme in a process requiring ATP as an energy source. Transfer of ubiquitin from E1 to the active site cysteine of a ubiquitin-conjugating enzyme E2 via a trans(thio)esterification reaction. The final step of the ubiquitylation cascade creates an isopeptide bond between a lysine of the target protein and the C-terminal glycine of ubiquitin. In general, this step requires the activity of one of the hundreds of E3 ubiquitin-protein ligases (often termed simply ubiquitin ligase). In the ubiquitination cascade, E1 can bind with dozens of E2s, which can bind with hundreds of E3s in a hierarchical way. It is preferred that a potential intracellular interaction partner is an E1, E2 or E3 enzyme involved in ubiquitination in case ubiquitination is determined in accordance with the invention. For example, a FRET-based sensor associated with the intracellular domain of the chimeric transmembrane receptor may be used for determining whether ubiquitination occurs. In case such FRET-based sensor is used, the potential intracellular interaction partner does not have to carry a label. FRET-based sensors for detecting ubiquitination are known in the art, for example, from Batters et al. (2010), PLOS one, Volume 5, Issue 2, e9008.; Ganesan et al., Proc Natl Acad Sci USA. 2006 103:4089-94.

Small Ubiquitin-like Modifier (or SUMO) proteins are a family of small proteins that are covalently attached to and detached from other proteins in cells to modify their function. SUMOylation is a post-translational modification involved in various cellular processes, such as nuclear-cytosolic transport, transcriptional regulation, apoptosis, protein stability, response to stress, and progression through the cell cycle. SUMO attachment to its target is similar to that of ubiquitin (as it is for the other ubiquitin-like proteins such as NEDD 8). A C-terminal peptide is cleaved from SUMO by a protease (in human these are the SENP proteases or Ulp1 in yeast) to reveal a di-glycine motif. SUMO then becomes bound to an E1 enzyme (SUMO Activating Enzyme (SAE)) which is a heterodimer. It is then passed to an E2 which is a conjugating enzyme (Ubc9). Finally, one of a small number of E3 ligating proteins attaches it to the protein. It is preferred that a potential intracellular interaction partner is an E1, E2 or E3 enzyme involved in SUMOylation in case SUMOylation is determined in accordance with the invention. For example, a FRET-based sensor associated with the intracellular domain of the chimeric transmembrane receptor may be used for determining whether SUMOylation occurs. In case such FRET-based sensor is used, the potential intracellular interaction partner does not have to carry a label. FRET-based sensors for detecting SUMOylation are known in the art, for example, from Stankovic-Valentin et al. (2009) Methods Mol Biol. 2009; 497:241-51.; Liu et al., Anal Biochem. 2012 422:14-21.

Oxidation or reduction reactions (also referred to as redox reactions) are characterized by an increase/decrease of electrons or an increase/decrease in oxidation state by a molecule, atom, or ion. One preferred example for an oxidation is the disulfide bond formation described above. Other preferred examples include the detection of exogenous or endogenous reactive oxidizing species (ROS) such as peroxides or superoxide. Exogenous ROS are for example produced by radiation and generally harmful to cells. Endogenous ROS are for example produced in mitochondria and by the NADPH oxidase complex. Endogenous ROS may also act as a second messenger in signal transduction processes. It is preferred that a potential intracellular interaction partner is a reactive oxygen specis in case such species are determined in accordance with the invention. For example, a sensor based on a cyclic permutated fluorescent protein associated with the intracellular domain of the chimeric transmembrane receptor may be used for determining whether oxidation or reduction via reactive oxygen species occurs. In case such cyclic permutated fluorescent protein-based sensor is used, the potential intracellular interaction partner does not have to carry a label. A sensor based on a cyclic permutated fluorescent protein for detecting hydrogen peroxide induced oxidation is known in the art, for example, from Belousov et al. Nat. Methods, 3 (2006), pp. 281-286.

Nitrosylation, specifically S-nitrosylation, involves the covalent incorporation of a nitric oxide moiety into thiol groups, to form S-nitrosothiol (SNO). S-nitrosylation is achieved through the non-catalysed chemical modification of a protein residue (see Martinez-Ruiz and Lamas, Cardiovasc Res (2004) 62 (1): 43-52). The reverse reaction, denitrosylation can be catalysed by the S-Nitrosoglutathione reductase. It is preferred that a potential intracellular interaction partner is nitric oxygen in case such species are determined in accordance with the invention. For example, a FRET-based sensor associated with the intracellular domain of the chimeric transmembrane receptor may be used for determining whether nitrosylation occurs. In case such FRET-based sensor is used, the potential intracellular interaction partner does not have to carry a label. FRET-based sensors for detecting nitrosylation are known in the art, for example, from St Croix et al., Methods Enzymol. 2005; 396:317-26; St Croix et al., Curr Protoc Cytom. 2008 Chapter 12: Unit 12.13.

Nucleotide addition (such as ADP-ribosylation) is the introduction of one or more nucleotides into a protein. E.g. ADP-ribosylation can be produced by NAD+:diphthamide ADP-ribosyltransferase enzymes, which transfer the ADP-ribose group from nicotinamide adenine dinucleotide (NAD+) onto acceptors such as arginine, glutamic acid, or aspartic acid. Multiple groups of ADP-ribose moieties can also be transferred to proteins to form long branched chains, in a reaction called poly(ADP-ribosyl)ation. This protein modification is carried out by the poly ADP-ribose polymerases (PARPs), which are found in most eukaryotes, but not prokaryotes or yeast. It is preferred that a potential intracellular interaction partner is an enzyme involved in nucleotide addition in case nucleotide addition is determined in accordance with the invention. For example, a FRET-based sensor associated with the intracellular domain of the chimeric transmembrane receptor may be used for determining whether nucleotide addition occurs. In case such FRET-based sensor is used the potential intracellular interaction partner does not have to carry a label. FRET-based sensors for detecting lipidation are not commonly available, but can in principle be build by combining a substrate sequence (e.g., a histone), a specific ADP-ribose binding domain (e.g. the Af1521 macro domain; Karras et al., EMBO J. 2005 24:1911-20) with a donor and acceptor fluorophore. The general guiding principle how such a sensor should be designed are known in the art (Miyawaki Annu Rev Biochem. 2011 80:357-73; Dehmelt and Bastiaens Nat Rev Mol Cell Biol. 2010 11:440-52).

Adenylylation is the process in which adenosine-5′-monophosphate (AMP) is covalently attached to a protein, nucleic acid, or small molecule via a phosphodiester or phosphoramidate linkage. Most often, the AMP is derived from ATP, but in some bacterial adenylylation reactions NADP+ is the source. Similarly, deadenylylation is the process in which AMP is removed from the adenylylated molecule. The adenylylation/deadenylylation processes may provide regulatory control of enzyme activity, contribute to intermediate steps in individual enzymatic reaction mechanisms, or occur as intermediate steps along the biosynthetic pathway of cofactors. It is preferred that a potential intracellular interaction partner is an enzyme involved in adenylylation in case adenylylation is determined in accordance with the invention. For example, a FRET-based sensor associated with the intracellular domain of the chimeric transmembrane receptor may be used for determining whether adenylylation occurs. In case such FRET-based sensor is used the potential intracellular interaction partner does not have to carry a label. FRET-based sensors for detecting adenylylation could be build by combining a substrate sequence (e.g., Rab1), a specific AMP binding domain with a donor and acceptor fluorophore. A AMP binding activity is present in the deAMPylase SidD (Tan and Luo Nature. 2011 475:506-9.), which could conceivably be further improved by mutational disruption of the deAMPylase activity. The general guiding principle how such a sensor should be designed are known in the art (Miyawaki Annu Rev Biochem. 2011 80:357-73; Dehmelt and Bastiaens Nat Rev Mol Cell Biol. 2010 11:440-52).

Arginylation is the tRNA-dependent posttranslational addition of Arg onto proteins. Arginylation is mediated by arginyltransferase (ATE1), an enzyme present in all eukaryotic cells. A similar modification also exists in prokaryotes, where a homologous enzyme L/F transferase modifies proteins by addition of Leu and Phe. It is preferred that a potential intracellular interaction partner is an enzyme involved in arginylation in case arginylation is determined in accordance with the invention. For example, a FRET-based sensor associated with the intracellular domain of the chimeric transmembrane receptor may be used for determining whether arginylation occurs. In case such FRET-based sensor is used the potential intracellular interaction partner does not have to carry a label. FRET-based sensors for detecting arginylation are not commonly available, but could in principle be build by combining a substrate sequence (e.g., an ATE1 substrate peptide that can be arginylated at its N-terminus; Rai et al., PNAS 2005 102:10123), a UBR domain that recognizes N-terminal arginines (Matta-Camacho et al., Nat Struct Mol Biol. 2010 17:1182-7.) with a donor and acceptor fluorophore. The general guiding principle how such a sensor should be designed are known in the art (Miyawaki Annu Rev Biochem. 2011 80:357-73; Dehmelt and Bastiaens Nat Rev Mol Cell Biol. 2010 11:440-52).

Isomerization of proline is the conversion of L-proline to D-proline and vice versa through a planar transition state, where the tetrahedral α-carbon becomes trigonal as a proton leaves the L-proline. The process is catalyzed by a peptidylprolyl isomerase. It is preferred that a potential intracellular interaction partner is a peptidylprolyl isomerase in case isomerization of proline is determined in accordance with the invention. For example, a FRET-based sensor associated with the intracellular domain of the chimeric transmembrane receptor may be used for determining whether isomerization of proline occurs. In case such FRET-based sensor is used the potential intracellular interaction partner does not have to carry a label. FRET-based sensors for detecting isomerization of proline are not commonly available, but could in principle be build by combining a substrate domain (e.g., the SH2 domain of the kinase Itk) with a donor and acceptor fluorophore. Isomerization of proline leads to a conformational change in this domain (Min et al., Front Biosci. 2005 10:385-97. Brazin et al., Proc Natl Acad Sci USA. 2002 99:1899-904.), which can in principle be detected via strategically positioned donor/acceptor fluorophores or a cyclic permuted fluorescent protein. The general guiding principle how such a sensor should be designed are known in the art (Miyawaki Annu Rev Biochem. 2011 80:357-73; Dehmelt and Bastiaens Nat Rev Mol Cell Biol. 2010 11:440-52).

Instead of the above-discussed FRET-based sensors also analogous BRET-based sensors may be employed.

In another preferred embodiment of the method of the invention, the reaction between a chimeric transmembrane receptor and an intracellular interaction partner thereof is an interaction between a chimeric transmembrane receptor and an intracellular interaction partner thereof, whereby the intracellular interaction partner is an intracellular binding partner.

In other words said preferred embodiment relates to a method for determining whether an interaction occurs between a chimeric transmembrane receptor and an intracellular binding partner thereof within a cell, said method comprising the steps of: a. providing a cell comprising: i. at least two distinct chimeric transmembrane receptors each comprising: (a) an extracellular binding domain, (b) a transmembrane domain, and (c) an intracellular domain, wherein said at least two transmembrane receptors are distinct in that (i) at least two of the domains (a), (b) and (c) are of different origin, (ii′) in that said extracellular binding domain of each of said at least two transmembrane receptors specifically interacts with a different extracellular compound, and (iii′) in that said intracellular domain of each of said at least two transmembrane receptors is different; and ii. one or more different potential intracellular binding partners that (i′) for step b.i. or step b.ii.(i′) or (ii′) are labelled with first labels; or (ii′) for step b.ii.(iii′) are unlabelled; b. contacting the cell with at least two different extracellular compounds, wherein each of said at least two extracellular compounds is bound to a surface i. in different areas of the same support, and/or ii. on different supports, (i′) wherein each support and its cognate transmembrane receptor form a complex that is labelled with a second label, (ii′) wherein each support can be distinguished by its shape and/or size, and/or (iii′) wherein in each support and its cognate transmembrane receptor the intracellular domain is labelled with a label or a pair of labels which is capable to indicate the binding of one or more potential intracellular binding partners of step a.ii. to the intracellular domain; and c. detecting i. said first label in step b.i.; and/or ii. said first label and second label, shape and/or size in step b.ii.; wherein i. for step c.i. the presence of a signal of said first label in (an) area(s) comprising the cognate extracellular compound and ii. for step c.ii.(i′) the presence of co-localized signals of said first and second label(s), for step c.ii.(ii′) co-localization of said first signal with said support, and for step c.ii.(iii′) a detectable conformational change of the label or a detectable energy transfer between the pair of labels is indicative of an interaction of a potential intracellular binding partner with a distinct chimeric transmembrane receptor.

A “potential intracellular binding partner” according to the invention may be any molecule or complex of the same or different molecules being labeled with a first label and may or may not be capable of interacting with the intracellular domain of a chimeric transmembrane receptor. In other words, the term “potential intracellular binding partner” in its broadest form embraces candidate ligands. For example, the capability of a given potential intracellular binding partner as candidate ligand to interact with a variety of different intracellular domains of different chimeric transmembrane receptors can be tested. In other words, different “bait” domains are present in the cytoplasm as part of transmembrane receptors, whereas as “prey” a candidate ligand is used. Molecules that can be labeled and thus used as potential intracellular binding partner may be molecules endogenously occurring in the cell or molecules not endogenously occurring in the cell. For example, molecules include but are not limited to peptides, polypeptides, lipids, nucleic acid molecules, small molecules, prodrugs, drugs, second messengers or metabolites. Preferably, the potential intracellular binding partner is a peptide or a polypeptide. It is understood that in accordance with the method of the invention more than a single potential intracellular binding partner molecule is present within the cell. Preferably, the cell comprises a multitude of potential intracellular binding partners of a kind such as, e.g. at least (for each value) 100, 250, 500, 1000, 2000, 3000, 4000, 5000, 10000, 50000, 100000, 10000000 or at least 100000000 potential intracellular binding partners of a kind. As is known in the art (Molecular Cell Biology. 4th edition. Lodish H, Berk A, Zipursky S L, et al. New York: W. H. Freeman; 2000. Section 1.2 The Molecules of Life), about 108 molecules of an abundant protein like actin are estimated to be present per cell.

The choice of the potential intracellular binding partners of the invention depends on the specific problem to be addressed in that intracellular domains of the chimeric transmembrane receptors have to be chosen that are known or are not known to be cognate to said potential intracellular binding partner. For example, if the interaction of the partners of a known interaction pair is to be determined, monitored or quantified in dependency of the status of said cell, the cell must comprise a chimeric transmembrane receptor whose intracellular domain represents or comprises the known interaction partner of the potential intracellular binding partner. On the other hand, if the cell is used, e.g., in a screening setup for identifying (so far unknown) interaction partners it is conceivably not necessary to have chimeric transmembrane receptors cognate to the potential intracellular binding partner, since only through screening the test agent's, i.e. the potential intracellular binding partner's interaction capacity and specificity, an interaction pair relationship between an potential intracellular binding partner and a chimeric transmembrane receptor may be established. Also envisaged is a combination of the above in the same cell, i.e. the presence of chimeric transmembrane receptors known to interact with the one or more potential intracellular binding partners and transmembrane receptors whose interaction capacity to said one or more potential intracellular binding partners is not known, i.e. is to be evaluated.

Within said preferred embodiment it is more preferred that the method is for determining whether an interaction occurs between a chimeric transmembrane receptor and an intracellular binding partner thereof within a cell, said method comprising the steps of: a. providing a cell comprising: i. at least two distinct chimeric transmembrane receptors each comprising: (a) an extracellular binding domain, (b) a transmembrane domain, and (c) an intracellular domain, wherein said at least two transmembrane receptors are distinct in that (i) at least two of the domains (a), (b) and (c) are of different origin, (ii′) in that said extracellular binding domain of each of said at least two transmembrane receptors specifically interacts with a different extracellular compound, and (iii′) in that said intracellular domain of each of said at least two transmembrane receptors is different; and ii. one or more different potential intracellular binding partners that are labelled with first labels; b. contacting the cell with at least two different extracellular compounds, wherein each of said at least two extracellular compounds is bound to a surface i. in different areas of the same support, and/or ii. on different supports, wherein each support and its cognate transmembrane receptor (i′) form a complex that is labelled with a second label and/or (ii′) wherein each support can be distinguished by its shape and/or size; and c. detecting i. said first label in step b.i.; and/or ii. said first label and second label, shape and/or size in step b.ii.; wherein i. for step c.i. the presence of a signal of said first label in (an) area(s) comprising the cognate extracellular compound and ii. for step c.ii. the presence of co-localized signals of said first and second label(s) or co-localization of said first signal with said support is indicative of an interaction of an potential intracellular binding partner with a distinct chimeric transmembrane receptor.

According to another preferred embodiment of the method of the invention, the detection of said first and/or said first and second labels, shape and/or size in step c. is effected over a period of time continuously or intermittently, thereby monitoring said reaction, preferably binding between said potential intracellular interaction, preferably binding partner(s) and said at least two chimeric transmembrane receptors. Preferably, said monitoring is a monitoring over time as explained in a preferred embodiment herein below.

The “period of time” is defined by the beginning of detection or an initial detection point and the end of detection or a final detection point in the case of continuous or intermittent detection, respectively. Said period of time is selected based on the specific reaction, preferably interaction that is to be determined and the specific investigative protocol used. Said period of time may comprise periods of continuous or intermittent detection of at least (for each value) 2, 5, 10, 15, 20, 25, 30 or at least 60 seconds. Also envisaged are shorter periods of time such as at least (for each value) 1, 5, 10, 15, 20, 25, 30 or at least 60 milliseconds as well as longer periods of time such as, e.g. at least (for each value) 2, 3, 4, 5, 10, 20, 30 or at least 60 minutes and equally preferred periods of at least (for each value) 2, 3, 4, 5, 6, 12, 24, 48, 72, 96 or at least 120 hours. In the case of intermittent detection, i.e. the detection at specific points in time within the specified period of detection, it is preferred that besides taking the initial detection point marking the beginning of the detection period and the final detection point marking the end of the detection period at least one further detection point, preferably at least (for each value) 2, 3, 4, 5, 6, 7, 8, 9 or at least 10 further detection points are taken. More preferred is that at least (for each value) 20, 30, 40, 50, 100, 200, 300, 400, 500, 1000 or at least 2000 further detection points are taken. Also preferred is that in case of intermittent detection the detection points are taken in uniform intervals. Nevertheless, it also envisaged that detection points can be associated with other parameters than time within the detection period. Such parameters may, e.g., be the change of concentration of a given substance that the cell used in accordance with the method of the invention is challenged with or the change of culture medium or any other parameter whose effect on the reaction(s), preferably being interaction(s), to be determined with the method of the invention is studied.

Detection over a period of time allows “monitoring” of a reaction (being preferably an interaction) over time, i.e. observing whether and when a reaction (being preferably an interaction) takes place or not, and/or whether and when the level of interaction decreases or increases, also allowing a quantitative analysis over time when a reaction, preferably a binding occurs.

According to a still further preferred embodiment of the method of the invention, the determining involves a quantification of said reaction(s), (being preferably interaction(s), between said potential intracellular interaction, preferably binding partner(s) and said at least two distinct chimeric transmembrane receptors.

Absolute quantification may, for example, be effected by measuring the signal intensity generated by said labels, i.e., first labels or first and second labels. Relative quantification of a reaction (being preferably an interaction) detected with the method of the invention is possible depending on the negative and/or positive controls used. Corresponding strategies for quantification based on, e.g., comparison of fluorescent signals are well-known to the skilled person and can be implemented without further ado. For example, if a reaction (preferably an interaction) is determined at a specific detection point the signal level can be compared to the signal level detected at a second specific point, thereby relative to the two detection points quantifying the reaction (preferably interaction) determined in accordance with the present invention. In the case that detection does not occur over time, quantification can be achieved by the use of suitable negative and/or positive controls. Thus, the method of the invention allows observing shifts in the reaction(s) (preferably interaction(s)), i.e., for example, when the equilibrium of bound potential intracellular interaction partners and unbound potential intracellular interaction, preferably binding partners shifts to either the bound or the unbound state.

According to a further preferred embodiment of the method of the invention, at least one of said at least two different extracellular compounds is bound to the surface of said same support more than once and in different areas to be covered by said cell; or wherein each of said at least two different extracellular compounds is bound to more than one of said different supports. This permits to spatially identify or determine said reaction (preferably interaction) between said potential intracellular interaction, preferably binding partner(s) and said at least two distinct chimeric transmembrane receptors. In other words, said reaction (preferably binding) can be assessed in dependency on the specific location within a cell.

In accordance with a further preferred embodiment of the method of the invention, said cell comprises at least two different potential intracellular interaction, preferably binding partners and wherein the intracellular domains of each of said at least two distinct chimeric transmembrane receptors specifically interact with one of said at least two different potential intracellular interaction, preferably binding partners, respectively.

This embodiment relates to cognate potential intracellular interaction, preferably binding partners, which term means that each of said at least two potential intracellular interaction, preferably binding partners specifically interacts with an intracellular domain of each said at least two distinct chimeric transmembrane receptors. In other words, the cell comprises a corresponding number of potential intracellular interaction, preferably binding partners and chimeric transmembrane receptors that are interaction, preferably binding partners, i.e. react, preferably bind specifically with each other but not to the intracellular domain of another chimeric transmembrane receptor used in accordance with the invention. A corresponding setup of the method of the invention allows studying whether, under certain conditions, a reaction, preferably a binding occurs or not between known interaction, preferably binding partners. The term “known interaction partners” refers to interaction partners which interact at least under one specific condition, but not necessarily under all conditions as they may occur in a living cell. The usefulness of such a setup is readily conceivable by the skilled person and has been described herein above. For example, the interactions of known interaction partners may be studied when changing the cellular context of the cell, e.g. by increasing or decreasing endogenous molecules within the cell or adding substances such as pharmaceutical agents.

In accordance with another preferred embodiment of the method of the invention, said different extracellular compounds do not specifically interact with an endogenous transmembrane receptor of the cell provided in step a.

An endogenous transmembrane receptor of the cell is a transmembrane receptor naturally-occurring in the cell.

According to still a further preferred embodiment of the method of the invention, said surface to which said different extracellular compounds are bound is a planar surface, preferably an array or a spherical surface, preferably a bead or a quantum dot.

The skilled person is well-aware of the meaning of the terms “planar” and “spherical” in the context of a surface of a support as used in the method of the invention. Depending on the experimental setup selected, i.e. whether the same support or different supports are used for immobilizing said at least two different extracellular compounds, the size of said planar or spherical support may vary as long as a cell or parts thereof come into contact with both of said at least two different extracellular compounds. An example of spherical support is a bead. Beads are known in the art and available from various manufacturers.

An array is in accordance with the invention a 2D array on a solid substrate (usually a glass slide or silicon thin-film cell) that assays large amounts of biological material, preferably by using high-throughput screening methods.

An example of spherical surface is a bead or an encased quantum dot. Beads are known in the art and available from various manufacturers. Quantum dots are small particles, or “nanoparticles”, of a semiconductor material, traditionally chalcogenides (selenides or sulfides) of metals like cadmium or zinc (CdSe or ZnS, for example), which range from 2 to 10 nanometers in diameter. The ability to precisely control the size of a quantum dot enables the manufacturer to determine the wavelength of the emission, which in turn determines the color of light the human eye perceives. Quantum dots can therefore be “tuned” during production to emit any colour of light desired. The ability to control, or “tune” the emission from the quantum dot by changing its core size is called in the art the “size quantisation effect”. The smaller the dot, the closer it is to the blue end of the spectrum, and the larger the dot, the closer to the red end. Dots can even be tuned beyond visible light, into the infra-red or into the ultra-violet.

In accordance with one preferred embodiment of the method of the invention, said different supports in step b.ii. can be taken up by said cell provided in step a.

It is understood in accordance with the invention that the preferably different supports are of a size and composition that allows uptake by the cell and/or does not endanger its viability. Preferably, the support is made of silica or organic polymers such as, e.g., polystyrene, with dimensions between 0.1 and 1 micrometer in size. The support can have any shape such as angled, e.g., rectangular, quadrate, triangular, or spherical such as, e.g., round or oval. Preferably, the shape of the supports is spherical as, e.g., for beads. Internalization of supports carrying immobilized different extracellular compounds can be achieved only by contacting said supports with the cell taking advantage of the natural ability of the cell to engulf extracellular entities. Said different supports may be directly seeded onto cells to stimulate their uptake or reversibly attached to a further planar support on to which a multitude of said different supports is linked in an area that is covered by a cell.

The different supports can be distinguished within the cell based on their size, shape and/or second label. For example, when the first label used emits a fluorescent signal, conventional or confocal fluorescence microscopy may be used to detect the potential intracellular interaction, preferably binding partner's label signal coupled with transmission microscopy to distinguish the size/shape of sufficiently different supports. Significantly differing shapes of (possibly) moving supports can be differentiated in confocal microscopy by their expected diffraction limited images. Methods like PALM (photo-activated localization microscopy) can increase the resolution and allow more precise determinations of distinct support shapes/sizes. Conventional fluorescence microscopy can also be employed when the internalized supports can (further) be distinguished by (a) fluorescent second label(s). Preferably, the different internalized supports are distinguishable by a second label, more preferred by a fluorescent second label and even more preferred by different fluorescent second labels. In line with the definitions given herein above, the second label may be fused or linked to the support, the extracellular compound and/or the chimeric transmembrane receptor. In other words, what matters is that the entity (complex) formed by support, extracellular compound and receptor is labeled. Preferably, the support is fluorescently labelled such as, e.g. quantum dots embedded in the polymer matrix of a bead. Alternatively, the support may be fluorescent per se such as a quantum dot. Also preferred is that the different supports are distinguishable by their size and fluorescent second label. Also, a combination of the two approaches is envisaged in accordance with the present invention.

The advantage of internalized supports is their ability to cover various spatial areas within a single cell over time. Integration of determined reactions, preferably interactions can provide temporal averages of the spatial pattern of reactions, preferably interactions with a small number of particles present in the cell.

According to a preferred embodiment of the method of the invention, said domains (a), (b) and (c) are synthetically designed domains or domains obtained from at least two different proteins of one or more species.

Means and methods for generating synthetically designed protein domains are well-established in the art; see, for example, Atherton, E.; Sheppard, R. C. (1989). Solid Phase peptide synthesis: a practical approach. Oxford, England: IRL Press; Albericio, F. (2000). Solid-Phase Synthesis: A Practical Guide (1 ed.). Boca Raton: CRC Press or Nilsson B L, Soellner M B, Raines R T (2005). “Chemical Synthesis of Proteins”. Annu. Rev. Biophys. Biomol. Struct.

The domains obtained from at least two different proteins of one or more species may be peptides being identical to a part of a protein or also complete proteins. In addition, dimers or multimers (such as trimers) of such peptides may be used.

According to a further preferred embodiment of the method of the invention, said extracellular binding domain and said transmembrane domain are inert with regard to triggering an intracellular response.

As discussed above, naturally-occurring transmembrane receptors have in general an extracellular binding domain having the ability to bind to a ligand and an intracellular domain having an activity that can be altered upon ligand binding. By these interactions, naturally-occurring transmembrane receptors can trigger an intracellular response. By contrast it is preferred that the extracellular binding domain and the transmembrane domain of the chimeric transmembrane receptor of the invention do not trigger an intracellular response. This may be, for example, achieved by introducing loss-of-function mutations into protein domains. Using inert extracellular binding domains and the transmembrane domains for the generation of the chimeric transmembrane receptor of the invention ensures that any intracellular response is solely triggered by the intracellular domain, which can be arbitrarily chosen for the specific application of this invention.

According to another preferred embodiment of the method of the invention, said extracellular binding domain is a protein binding domain, an antibody epitope, an antibody, an oligonucleotide binding domain, or a small molecule binding domain.

Within this list of extracellular binding domains particular preference is given to an antibody epitope.

An antibody epitope (also known as antigenic determinant in the art) is the part of an antigen that is recognized by an antibody. Epitopes are often used in proteomics and the study of other gene products. Using well-known recombinant DNA techniques genetic sequences coding for epitopes that are recognized by common antibodies can be generated and used for the generation of the chimeric transmembrane receptor of the invention.

A protein binding domain binds to an amino acid sequence, either being a (poly)peptide or a protein. The protein binding domain may itself be an amino acid sequence, such as a peptide.

The term “antibody” as used in accordance with the present invention comprises, for example, polyclonal or monoclonal antibodies. Furthermore, also derivatives or fragments thereof, which still retain the binding specificity, are comprised in the term “antibody”. Antibody fragments or derivatives comprise, inter alia, Fab or Fab′ fragments, Fd, F(ab′)2, Fv or scFv fragments, single domain VH or V-like domains, such as VhH or V-NAR-domains, as well as multimeric formats such as minibodies, diabodies, tribodies, tetrabodies or chemically conjugated Fab′-multimers (see, for example, Altshuler et al., 2010., Holliger and Hudson, 2005). The term “antibody” also includes embodiments such as chimeric (human constant domain, non-human variable domain), single chain and humanized (human antibody with the exception of non-human CDRs) antibodies. Various techniques for the production of antibodies and fragments thereof are well known in the art and described, e.g. in Altshuler et al., 2010. Thus, polyclonal antibodies can be obtained from the blood of an animal following immunisation with an antigen in mixture with additives and adjuvans and monoclonal antibodies can be produced by any technique which provides antibodies produced by continuous cell line cultures. Examples for such techniques are described, e.g. Harlow and Lane (1988) and (1999) and include the hybridoma technique originally described by Köhler and Milstein, 1975, the trioma technique, the human B-cell hybridoma technique (see e.g. Kozbor, 1983; Li et al., 2006) and the EBV-hybridoma technique to produce human monoclonal antibodies (Cole et al., 1985). Furthermore, recombinant antibodies may be obtained from monoclonal antibodies or can be prepared de novo using various display methods such as phage, ribosomal, mRNA, or cell display. A suitable system for the expression of the recombinant (humanized) antibodies or fragments thereof may be selected from, for example, bacteria, yeast, insects, mammalian cell lines or transgenic animals or plants (see, e.g., U.S. Pat. No. 6,080,560; Holliger and Hudson, 2005). Further, techniques described for the production of single chain antibodies (see, inter alia, U.S. Pat. No. 4,946,778) can be adapted to produce single chain antibodies specific for the target of this invention. Surface plasmon resonance as employed in the BIAcore system can be used to characterize the efficiency of phage antibodies for further optimization.

An oligonucleotide binding domain binds to a short single-stranded nucleic acid molecule, being preferably a short single-stranded nucleic acid molecule. The oligonucleotide binding domain may be a nucleic acid molecule or a (poly)peptide, such as an aptamer (e.g. DNA or RNA or XNA or protein aptamers). An oligonucleotide preferably consists of at least 10 and up to 30 nucleotides. Nucleic acid molecules, in accordance with the present invention, include DNA, such as cDNA or genomic DNA, and RNA. It is understood that the term “RNA” as used herein comprises all forms of RNA including non-functional RNA (i.e. biologically inactive RNA), mRNA, ncRNA (non-coding RNA), tRNA and rRNA. The term “non-coding RNA” includes siRNA (small interfering RNA), miRNA (micro RNA), rasiRNA (repeat associated RNA), snoRNA (small nucleolar RNA), and snRNA (small nuclear RNA). Preferably, embodiments reciting “RNA” are directed to synthetic RNA. At the same time, other forms of RNA, including the above mentioned specific forms, are deliberately envisaged in the respective embodiments. Furthermore included is genomic RNA, such as in case of RNA of RNA viruses.

Further included by oligonucleotide binding domains are nucleic acid mimicking molecules known in the art such as synthetic or semisynthetic derivatives of DNA or RNA and mixed polymers, both sense and antisense strands. They may contain additional non-natural or derivatized nucleotide bases, as will be readily appreciated by those skilled in the art. In a preferred embodiment the polynucleotide or the nucleic acid molecule(s) is/are DNA. Such nucleic acid mimicking molecules or nucleic acid derivatives according to the invention include phosphorothioate nucleic acid, phosphoramidate nucleic acid, 2′-O-methoxyethyl ribonucleic acid, morpholino nucleic acid, hexitol nucleic acid (HNA) and locked nucleic acid (LNA) (see, for example, Braasch and Corey, Chemistry & Biology 8, 1-7 (2001)). LNA is an RNA derivative in which the ribose ring is constrained by a methylene linkage between the 2′-oxygen and the 4′-carbon. Peptide nucleic acid (PNA), PNA chimera, term “derivatives” in conjunction with the above described PNAs, (poly)peptides, PNA chimera and peptide-DNA chimera are described in greater detail herein above.

A small molecule binding domain binds to a small organic or anorganic compound. A small molecule binding domain may be a (poly)peptide or a nucleic acid molecule, such as an aptamer (e.g. DNA or RNA or XNA or protein aptamers). The small molecule is preferably a low molecular weight (preferably <800 Daltons and more preferably <500 Daltons) organic compound. A small molecule may be for example a primary and secondary metabolite of a cell. Preferably for this invention, this small molecule would not elicit any biological function in cells other than binding to the small molecule binding domain.

According to a more preferred embodiment of the method of the invention, said oligonucleotide binding domain comprises or consists of one or more zinc finger domains, TAL repeats, helix-turn-helix domains, leucine zippers, winged helix domains, winged helix-turn-helix domains, helix-loop-helix domains, HMG-boxes, (mutant) restriction nucleases, PUF repeats, zinc-containing RNA binders, KH domains, RRM domains or RBD/RRM/RNP domains.

Within this list of oligonucleotide binding domains, zinc finger domains and TAL repeats are particularly preferred.

Zinc-finger proteins are small structural motifs that can coordinate one or more zinc ions to help stabilize their folds. They can be classified into several different families and typically function as interaction modules that can specifically bind DNA and RNA molecules as well as peptides or polypeptides or small molecules. The skilled person in the art is aware of means and methods to modify known zinc-finger DNA binding domains so that a specific nucleic acid molecule is specifically bound (Pavletich and Pabo, Science, (1991) 252(5007):809-817). Preferably, the zinc-finger DNA binding domain belongs to the Cys2His2-like fold group, such as e.g., of the murine transcription factor Zif268. Also preferred is that the Zinc-finger DNA binding domain comprises 3 to 6 individual zinc-finger motifs in order to bind nucleic acid molecules as extracellular compounds ranging from 9 basepairs to 18 basepairs in length. Also preferred is that the Zinc-finger DNA binding domain is modified to be redox insensitive and therefore also applicable in the oxidative conditions found outside cells.

TAL repeats (Transcription activator-like repeats) are derived from transcription factors of Xanthomonas bacteria. Transcription activator-like repeats can be quickly engineered to bind practically any desired DNA sequence (Boch, Jens (February 2011). “TALEs of genome targeting”. Nature Biotechnology 29 (2): 135-6.).

According to a preferred embodiment of the method of the invention, (i) said extracellular binding domain and said transmembrane domain and/or (ii) said transmembrane domain and said intracellular domain are connected by a linker sequence, said linker being preferably one or more (biologically inert) immunoglobulin domains, a flexible domain protein linker, such as a Glycine-Serine linker, or a rigid linker, such as a helix-forming rigid linker.

The term “linker” as used in accordance with the present invention relates to a sequence of amino acids (i.e. peptide linkers), which may separate the transmembrane domain form the extracellular binding domain and/or the intracellular domain.

Peptide linkers as envisaged by the present invention may be (poly)peptide linkers of at least 1 amino acid in length. Preferably, the linkers are 1 to 100 amino acids in length. More preferably, the linkers are 5 to 50 amino acids in length and even more preferably, the linkers are 10 to 20 amino acids in length.

The peptide linker is preferably a flexible domain protein linker using e.g. the amino acids alanine and serine or glycine and serine (i.e. a Glycine-Serine linker). Preferably the Glycine-Serine linker has the sequence (Gly4Ser)4, (SEQ IDE NO: 1) or (Gly4Ser)3 (SEQ ID NO: 2).

In particular rigid linkers may be advantageous to sterically separate transmembrane domain form the extracellular binding domain and/or the intracellular domain. A preferred example of a rigid linker is a helix-forming rigid linker, such as the helical linker (EAAAK)n (SEQ ID NO: 3), wherein n is an integer between 2 and 5.

An immunoglobulin domain is a protein domain consisting in general of a 2-layer sandwich of between 7 and 9 antiparallel β-strands arranged in two β-sheets with a Greek key topology. The immunoglobulin domains are preferably biologically inert. Biologically inert immunoglobulin domains do not trigger a cellular response. A particular example of an immunoglobulin domain is the titin Ig domain 127 (SEQ ID NO: 4).

According to a further preferred embodiment of the method of the invention, said transmembrane domain is an artificially designed transmembrane domain, an alpha-helical transmembrane domain of a single-span membrane protein, a transmembrane domain of a growth factor receptor, or a multiple-pass transmembrane domain.

Within this list of transmembrane domains an artificially designed transmembrane domain and an alpha-helical transmembrane domain of a single-span membrane protein are particularly preferred.

The alpha-helical transmembrane domain of a single-span membrane protein is preferably chosen from the single membrane spanning receptors of the receptor tyrosine kinase family. For example transmembrane receptors from the EGFR subfamily (e.g. Erbb2, Erbb3), the PDGF subfamily (e.g. PDGFR-alpha, PDGFR-beta), the FGF subfamily (e.g. FGFR1, FGFR2, FGFR3, FGFR4) or the TRK subfamily (e.g. TrkA, TrkB, TrkC) are selected.

As defined above the transmembrane domain may be any three-dimensional protein structure which is thermodynamically stable in a cell membrane and spans the cell membrane. Such domain may also be artificially designed based on molecular modelling. Naturally-occurring transmembrane protein may be callssified into four types (Harvey Lodish etc.; Molecular Cell Biology, Sixth edition, p. 546). Types I, II, and III are single pass molecules, while type IV are multiple pass molecules. Type I transmembrane proteins are anchored to the lipid membrane with a stop-transfer anchor sequence and have their N-terminal domains targeted to the ER lumen during synthesis (and the extracellular space, if mature forms are located on plasmalemma). Type II and III are anchored with a signal-anchor sequence, with type II being targeted to the ER lumen with its C-terminal domain, while type III have their N-terminal domains targeted to the ER lumen. Type IV is subdivided into IV-A, with their N-terminal domains targeted to the cytosol and IV-B, with an N-terminal domain targeted to the lumen. The implications for the division in the four types are especially manifest at the time of translocation and ER-bound translation, when the protein has to be passed through the ER membrane in a direction dependent on the type. Alpha-helical transmembrane protein is the major category of transmembrane proteins. In humans, 27% of all proteins have been estimated to be alpha-helical membrane proteins (Almén et al. (2009). BMC Biol. 7: 50).

According to another preferred embodiment of the method of the invention, the intracellular domain is or comprises a protein binding domain, a small molecule binding domain, a oligonucleotide binding domain, or a sensor construct, an enzyme, an antibody epitope, a chelator.

A binding domain, a small molecule binding domain, an antibody epitope, an oligonucleotide binding domain are defined herein above. In accordance with this embodiment the small molecule/protein/oligonucleotide binding domain is preferably an allosterically regulated small molecule/protein/oligonucleotide binding domain. Allosteric regulation is the regulation of the domain by binding an effector molecule at the domains allosteric site (that is, a site other than the active site of the intracellular domain). Effectors that enhance the protein's activity are referred to in the art as allosteric activators, whereas those that decrease the protein's activity are in the art as allosteric inhibitors. In accordance with this embodiment the small molecule/protein/oligonucleotide binding domain is preferably regulated by an enzymatic activity, such as a kinase activity. Vice versa, the reaction, preferably interaction of the intracellular domain of a chimeric receptor with a potential intracellular interaction, preferably binding partner can be regulated by an additional allosteric binding site on either of the two potential interaction, preferably binding partners.

A sensor construct is a sensitive biological element (e.g. cell receptors, enzymes, antibodies, nucleic acids, etc.), a biologically derived material or biomimetic component that is capable to interact with (e.g. reacts with or recognizes) the analyte under study. The sensor construct is furthermore capable to interact with a transducer or the detector element. The transducer or the detector element (e.g. working in a physicochemical, optical, piezoelectric, or electrochemical, way) transforms the signal resulting from the interaction of the analyte with the sensor construct into another signal (i.e., transduces) that can be measured and quantified. A particular example is a FRET (Forster (Fluorescence) resonance energy transfer)-based sensor construct. To this end, modifications of FRET such as BRET (bioluminescence resonance energy transfer) are also envisaged.

An enzyme is a complex protein that is produced by living cells and catalyzes specific biochemical reactions at body temperatures. Enzymes may be classified by the type of reaction they catalyze: (1) oxidation-reduction, (2) transfer of a chemical group, (3) hydrolysis, (4) removal or addition of a chemical group, (5) isomerization, and (6) binding together of substrate units (polymerization).

A chelator is a compound that binds metal ions from solutions, such as the cytosol. The compound may be a chemical, such as EDTA or a protein, such as chlorophyll of hemoglobin.

The present invention also relates to a kit comprising a. the cell as defined in step a. of the method of the invention and the extracellular compounds as defined in step b. of the method of the invention; and/or b. at least two different extracellular compounds as defined in step b. of the method of the invention and nucleic acid molecules encoding the one or more potential intracellular interaction, preferably binding partners as defined in step a.ii. of the method of the invention and the at least two distinct chimeric transmembrane receptors as defined in step a.i. of the method of the invention.

Any of the entities or complexes of entities that are employed in the method of the invention such as, e.g., the extracellular compounds, the chimeric transmembrane receptors or the domains thereof, the potential intracellular interaction, preferably binding partners, fluorescent polypeptides, in the form of the actual polypeptide or in the form of nucleic acid molecules encoding the latter, e.g., as part of expression vectors, the cell, the supports, suitable coatings for the support and/or linkers may be comprised in a kit such as, e.g., defined above.

The components of the kits may be packaged in aqueous media or dried, e.g. lyophilized, where possible. Preferably, the above-mentioned entities are comprised in a kit according to the invention as separate components. Accordingly, the various components of the kit may be packaged in one or more containers such as one or more vials, test tubes, flasks, bottles or other container means. The latter may, in addition to the components, comprise preservatives or buffers for storage, media for maintenance and storage, e.g. cell media, DMEM, MEM, HBSS, PBS, HEPES, hygromycin, puromycin, Penicillin-Streptomycin solution, gentamicin inter alia. Advantageously, the kit further comprises instructions for use of the components allowing the skilled person to conveniently work, e.g., various embodiments of the invention. Any of the components may be employed in an experimental setting. The kits according to the invention may also include a means for containing the kit components in close confinement. Such containers may include cardboard or plastic containers into which the desired components are retained.

The present invention furthermore relates to a chimeric transmembrane receptor, comprising: (a) an extracellular binding domain comprising an epitope which is connected to the transmembrane domain of (b) via four repeats of the titin Ig domain 127, (b) a transmembrane domain comprising a single transmembrane domain obtained from the platelet-derived growth factor receptor, and (c) an intracellular domain which comprises (i) a protein of interest (being, e.g., a protein acting as bait or a potential interaction, preferably binding partner) and (ii) a fluorescent protein.

Features (a), (b) and (c) of the chimeric transmebrane receptor may either be in a N-terminus to C-terminus order or in a C-terminus to N-terminus order, as long as the localization of the extracellular and intracellular domains are correct with respect to insertion of the mature receptor into the plasma membrane of the cell. It is preferred that the chimeric transmembrane receptor comprises SEQ ID NO: 5 or 6. SEQ ID NOs: 5 and 6 comprise the amino acid sequence of the chimeric transmembrane receptor with exception to the protein of interest. Any protein of interest can be added to the C-terminus of SEQ ID NO: 5 and 6 thereby arriving at the complete chimeric transmembrane receptor. In principle any epitope tag can be recognized by a suitable (preferably high affinity) antibody. The epitope of SEQ ID NO: 5 are three consecutive VSVG-Tags and in SEQ ID NO: 6 an HA-Tag. At least one of said chimeric transmembrane receptors is preferably used in the method and kit of the invention. Such a chimeric transmembrane receptor was also used in the examples herein below and has the amino acid sequence of SEQ ID NO: 7.

In accordance with a preferred embodiment of the kit and methods of the invention (a) said extracellular binding domain is/are an epitope and said at least one, preferably at least two compound(s) is/are an antibody recognizing said epitope; (b) said extracellular binding domain(s) is/are DNA binding domain(s) and said compound(s) is/are cognate DNA; or (c) said extracellular binding domain(s) is/are antibody/ies and said compound(s) is/are or comprise(s) epitope(s) recognized by said antibody/ies.

The figures show:

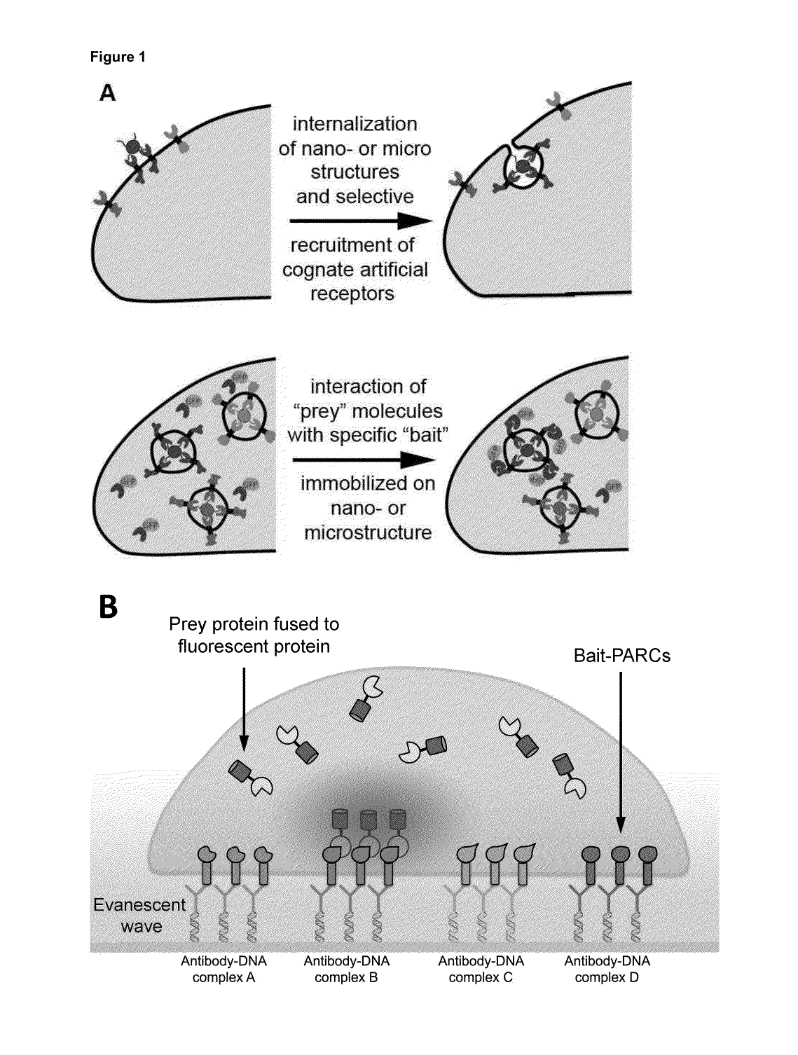

FIG. 1 Principle of live cell multiplex biosensors. Bait-presenting artificial receptor constructs (bait-PARCs) are expressed on the cell surface and recruited by cognate immobilized extracellular compounds on fiduciary marks. A) In one implementation, the fiduciary marks are nano- or microstructures, which are internalized by the cell. These fiduciary marks are mobile within the cytoplasm. B) In another implementation, the bait-PARCs are recruited to immobilized extracellular compounds, such as for example antibody-DNA complexes, which are arrayed on a structured surface. In both implementations, these fiduciary marks enable the localization of bait proteins within individual cells. Potential intracellular interaction partners, such as soluble, fluorescent proteins of interest, act as prey. The interaction between bait and prey and the identity of fiduciary marks is monitored by a suitable method.

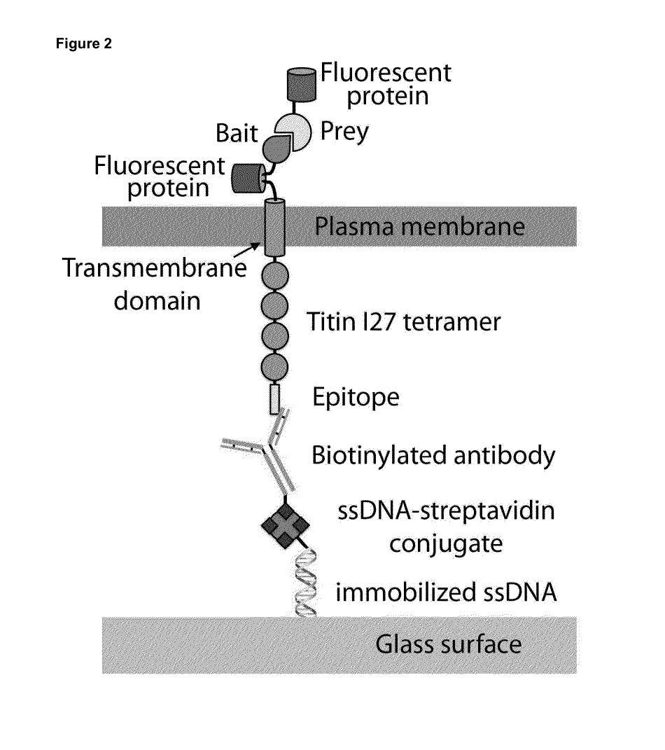

FIG. 2 Schematic illustration of bait presenting artificial receptor constructs (bait-PARCs) and immobilized antibodies. Oligonucleotide arrays on glass substrates are functionalized with antibodies by DNA-directed immobilization (DDI). The extracellular region of bait-PARCs contains an epitope, which binds to the immobilized antibody. The intracellular region of bait-PARCs is composed of a bait protein fused to a fluorescent protein. A cytosolic interaction partner acting as prey is fused to a spectrally distinct fluorescent protein.

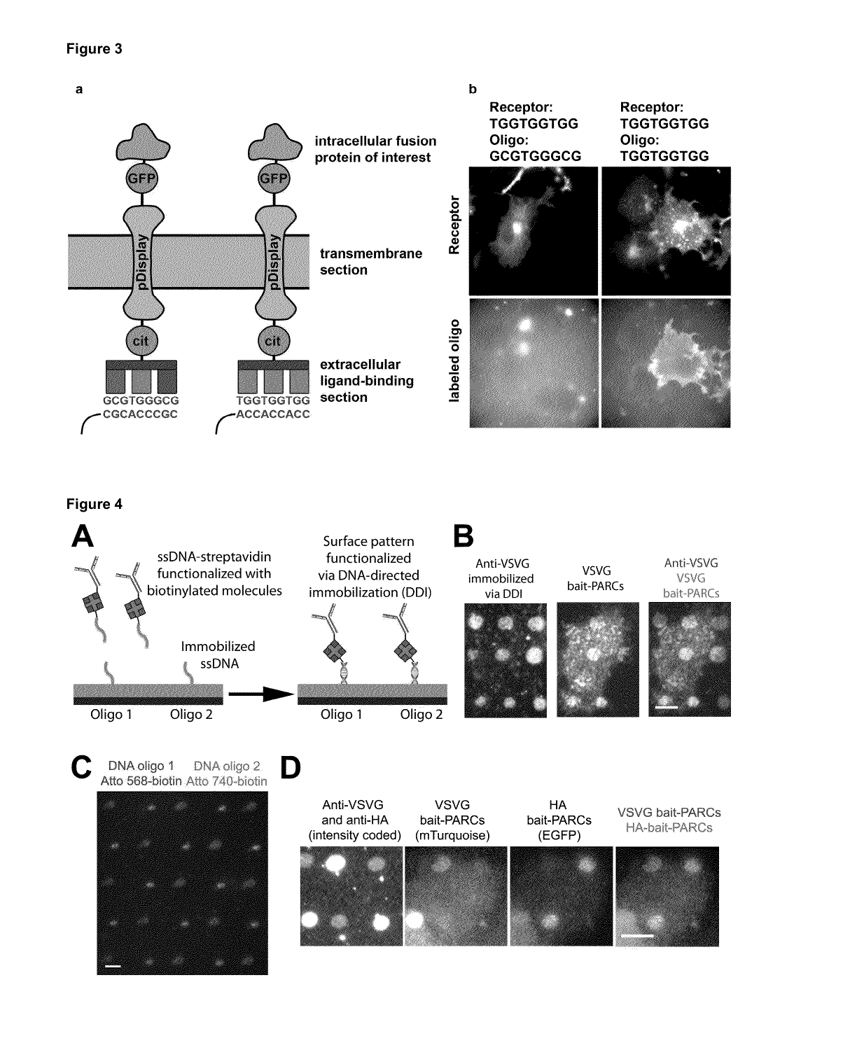

FIG. 3 Zinc-finger based bait-PARCs that directly recognize specific DNA sequences. a) Schematic for the design of two orthogonal artificial receptor-extracellular compound pairs based on the zinc-finger DNA interaction. b) Microscopic analysis of specific oligonucleotide binding to cells expressing the receptor variant 2. Only the oligonucleotide with the sequence TGGTGGTGG is recognized by its cognate receptor (panel at lower right). The oligonucleotide with the sequence GCGTGGGCG is not recognized by this receptor (panel at lower left). Conversely, the receptor specific for oligonucleotide GCGTGGGCG specifically only recognizes its cognate oligonucleotide (not shown).

FIG. 4 Micro-patterning of bait proteins in living cells. A: Schematic of the application of DNA-directed immobilization (DDI) to generate arrays of immobilized antibodies. B: Bait-PARCs displaying VSVG epitope tags are recruited to anti-VSVG functionalized surface patterns within the plasma membrane of COS7 cells. Scale bar: 5 μm C: Selective surface functionalization via DDI. Two distinct capture-oligonucleotides were immobilized on an activated glass surface via dip pen nanolithography. Two complementary, streptavidin-modified oligonucleotides were saturated with spectrally separable biotinylated fluorophores and functionalized with distinct antibodies. Simultaneous hybridization reveals site-specific direction of the respective complexes to complementary, immobilized oligonucleotides. Scale bar: 5 μm D: Checkerboard patterns of two distinct antibodies, anti-VSVG and anti-HA, were generated via DDI. The identification of these antibodies is based on intensity coding of Atto 740 fluorophores. Two distinct bait-PARCs, which display the corresponding peptide epitopes (HA and VSVG-Tags) in their extracellular region were co-expressed in COS7 cells and enriched in cognate antibody microstructures. Scale bar: 10 μm.

FIG. 5 Basis for bead-based multiplex biosensors. a) Schematic for micro-patterning of receptors in living cells via surface-immobilized submicrometer size streptavidin-functionalized beads. Beads can either be immobilized on the cell substrate or can be allowed to be internalized into cells b) Recruitment of a kinase-dead growth factor receptor to surface immobilized beads in living cells. The top panel shows accumulation of the receptor at submicrometer-sized regions within the cellular plasma membrane. These locations correspond to the position of immobilized, growth factor coated beads in the lower panel (all micrographs were obtained via TIRF microscopy). c) Recruitment of growth factor receptors to mobile beads in living cells. Tracks indicate bead mobility within living cells.

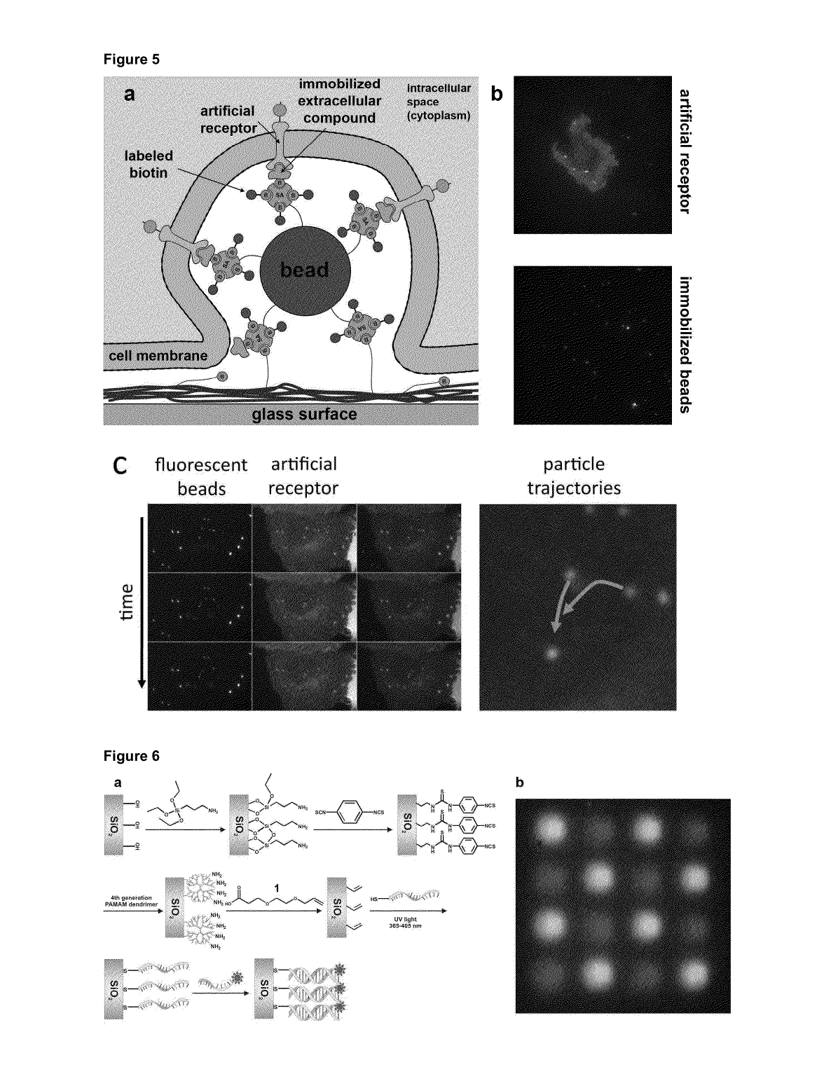

FIG. 6 Microstructuring by immobilization of DNA oligonucleotides on a glass surface via photolithography. a) Schematic of the surface modification procedure. b) Fluorescent micrograph of Alexa-488 and Alexa-568 labeled oligonucleotides, which interact with sequentially written complementary oligonucleotides (structure size: approx. 2 μm).

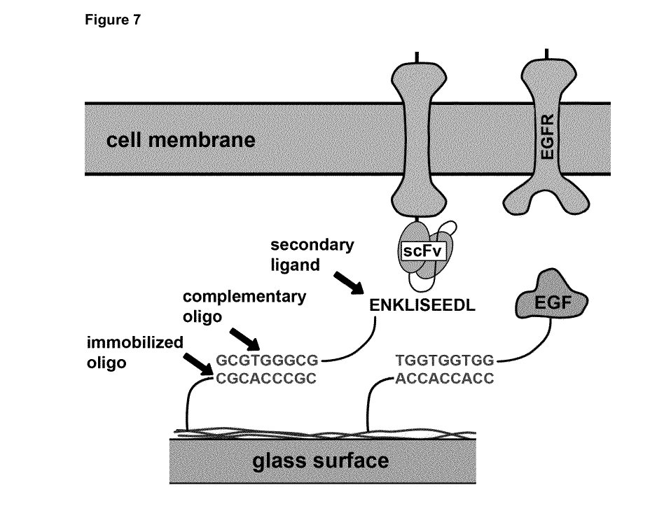

FIG. 7 Indirect coupling via secondary extracellular compounds. Due to its chemical stability, DNA is compatible with many linking strategies and therefore the first choice for surface functionalization. Oligo functionalized surfaces can be converted into many other biomolecule arrays via DNA-directed immobilization (DDI).

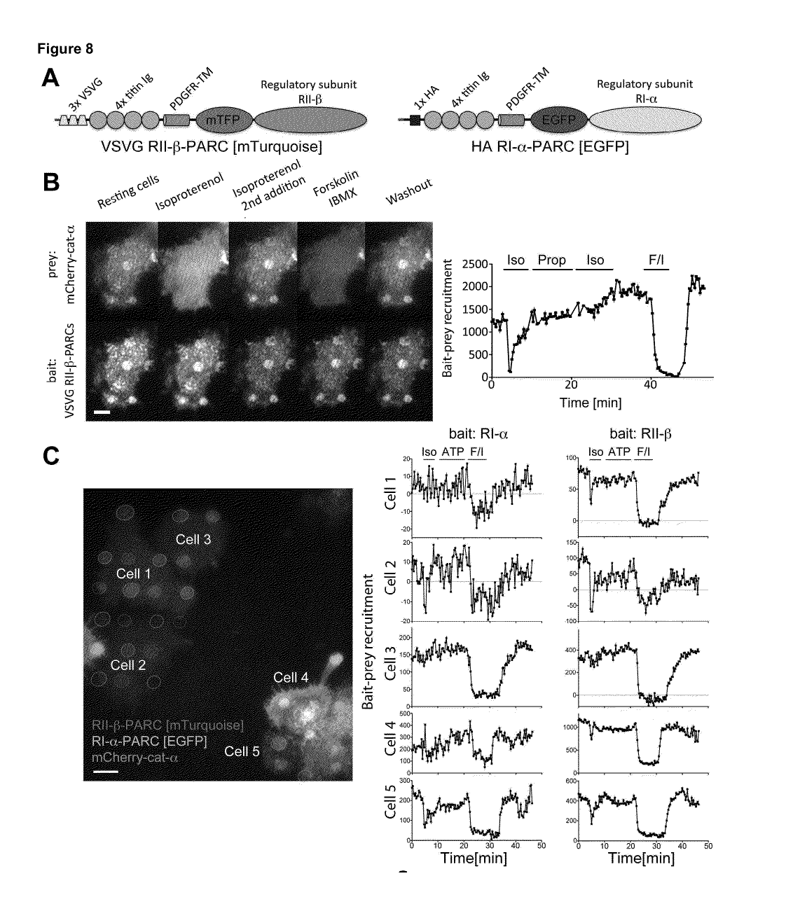

FIG. 8 Monitoring protein reaction dynamics inside individual cells. A: Domain structures of bait-PARCs to measure PKA subunit interactions. B: A bait-PARC containing the regulatory domain RII-β of PKA was co-expressed with the cytosolic prey protein (mCherry-cat- [symbol] to monitor their interaction dynamics inside living cells. Left: Recruitment of the prey to bait microstructures before and after pharmacological perturbation. Right: Derived prey recruitment kinetics (Iso: Isoproterenol; Prop: Propranolol; F/I: forskolin+IBMX; see Supplementary Methods for details). Scale bar: 5 μm. c: Two distinct regulatory domains on bait-PARCs were co-expressed together with the prey protein mCherry-cat- [symbol] . Left: Image of a representative experiment depicting cells grown on a DNA-immobilized antibody array. The checkerboard pattern of antibodies is overlayed with magenta (anti-HA) or cyan (anti-VSVG) circles. Right: The recruitment of prey proteins to the two distinct bait proteins was monitored in individual microstructured spots via TIRFM during pharmacological perturbation (Iso: Isoproterenol; ATP: adenosine triphosphate; F/I: forskolin+IBMX). Scale bar: 10 μm.

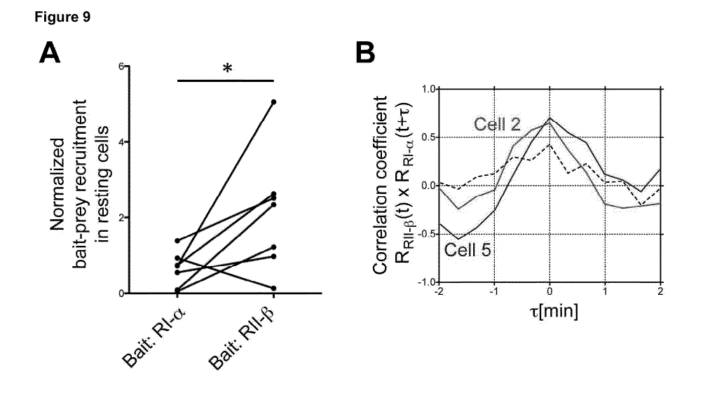

FIG. 9 Detection of relations between multiple protein reactions inside individual cells. A: Paired measurements of the interaction between the prey protein and the two bait proteins in individual, resting cells. The two experimental groups are significantly different (p<0.05; Wilcoxon signed rank test, n=7 cells from 3 independent experiments). B: Temporal cross-correlation profiles for the response of the two distinct regulatory subunits during β-adrenergic receptor stimulation. The cross-correlation is calculated from the recruitment kinetics and plotted as a function of the time shift τ. The red and blue lines show correlation profiles for two individual cells, the black line shows the average profile of 7 cells.

The examples illustrate the invention.

### EXAMPLES

### Example 1

### Principle of Live Cell Multiplex Biosensors of the Invention

Multiple arbitrary proteins of choice, hereafter referred to as bait, are localized inside living cells via distinguishable fiduciary marks, such as quantum dots, labeled nanospheres, color-coded beads, or spatially separated surface structures. Localization of these proteins to the fiduciary marks is achieved by the interaction of bait presenting artificial receptor constructs (bait-PARCs) with surface-linked extracellular compounds (FIG. 1). Based on these fiduciary marks, multiple protein baits are distinguished to enable measurements of multiple interactions with intracellular, fluorescently labeled binding partners. The measurement of such interactions is both spatially and temporally resolved and performed in living cells. These measurements are used to detect the state of multiple protein-interactions and protein reactions as well as their temporal changes and spatial organization in individual living cells.

First, a set of distinct molecules, such as antibodies, peptides or DNA oligonucleotides, is immobilized on a solid support. This solid support structure could for example be a library of color-coded beads or a 2-dimensional, addressable array similar to a gene chip. Next, cells that express multiple, different bait-PARCs interact with these surfaces. The bait-PARCs are composed of three sections: a) an extracellular module that specifically interacts with one of the surface-linked molecules, b) a transmembrane module that is essential for insertion of the receptor into the cellular plasma membrane, and c) an intracellular, arbitrary protein segment that functions as bait. Both the surface-linked molecule-binding module and the intracellular bait are unique for each of the different bait-PARCs. Bait-PARCs will be selectively recruited to the surface functionalized with a cognate molecule. The individual functionalized surfaces are distinguished by specific properties, such as their size, shape or photophysical characteristics. These surfaces, which are linked to bait proteins, form a set of distinguishable probes inside a living cell.

Another, fluorescently labeled, soluble protein of interest is expressed in the same cell and functions as prey. Its interaction with this set of probes can be monitored via microscopic analysis—for example via tracking and spectral analysis of internalized nano- or microstructures by fluorescence microscopy, or via excitation of surface associated fluorescence via the evanescent wave of a total internal reflection (TIRF) microscope (FIG. 1).

In combination, these components represent a live cell multiplex biosensor, capable of directly measuring multiple protein reactions inside an individual, living cell. In previous studies, interactions between a native receptor, CD4, and one of it's binding partners, Lck, was measured via a related approach (Schwarzenbacher et al., 2008). However, that approach does not address the general applicability to study protein interactions, and the technology involved is limited to measuring a single interaction in cells. In a second study, two co-stimulatory ligands were immobilized and the effect of their spatial organization on cell behavior and interactions with known binding partners was studied (Shen et al., 2008). In that study, the organization of the stimulatory ligands influenced the measurement of protein interactions and is thus not applicable in a broader, more general scope outside the observed biological application. In another study (Zamir et al., 2010), the interaction between “bait”-fused quantum dots and a soluble, fluorescently labeled “prey” was reported, however, the technical implementation, which required injection of tailor made functionalized quantum dots follows a distinct strategy and is not scalable to larger numbers of interaction pairs.

In contrast, this invention opens the way for simultaneous, time resolved measurement of multiple protein interactions in a single, individual cell. This unique feature of this invention enables extraction of detailed information about the correlations between protein reactions and thereby allows time-resolved analysis of protein network states in individual cells. Furthermore, in contrast to previous studies, our approach offers a general strategy that extends beyond measuring naturally occurring interactions between a wild-type receptor and a known interaction partner as in (Schwarzenbacher et al., 2008). Here, by constructing bait-presenting artificial receptor constructs, multiplex biosensors can be tailored to analyze the interaction between arbitrary pairs of proteins.

This principle also permits sensing the composition of the extracellular environment via readout of the live cell multiplex biosensor. This can be applied in biomedical research, clinical assay development or environmental monitoring of trace compounds that can elicit natural or engineered reactions in living cells. In particular, the simultaneous measurement of multiple protein interactions and protein reactions in an individual cell enables discrimination of multiple network states, which will allow sensitive and robust analysis readouts. Finally, by distinguishing different network states via multiplexed measurements, multiple components can be distinguished in complex extracellular assay mixtures.

### Example 2

### Implementations of Bait-PARCs

The kit of the present invention may be composed of three components: (i) bait-PARCs, (ii) a set of cognate surface-immobilized molecules and (ii) an intracellular soluble fluorescent molecule, termed prey. Also the method of the invention uses these three components.

### Bait-PARCs:

Bait-PARCs may be constructed containing a) an extracellular binding domain that binds specifically to functionalized surfaces, b) a transmembrane domain and c) an intracellular protein of interest termed bait (FIG. 2). The extracellular binding domain can be derived from a naturally occurring cell surface receptor, as long as neither the receptor, nor its cognate extracellular compound elicits a cellular response. Preferentially, it is derived from a non-receptor type interaction module, that does not elicit an intracellular response, such as an epitope recognized by an antibody, a DNA-binding protein (Wolfe et al., 2000) or an artificial single chain antibody (Holliger and Hudson, 2005). The sole purpose of the extracellular binding domain is to enable the recruitment of the bait-PARC to the surface-immobilized extracellular compound. The following examples illustrate these possibilities:

### Bait-PARCs Based on the Interaction Between a Surface-Immobilized Antibody and a Corresponding Epitope:

As a proof of concept, artificial receptors were constructed that can be patterned by surface-linked antibodys. These receptors contain the following features (FIG. 2): 1) an intracellular domain that contains a fluorescent protein fused to an arbitrary bait protein, 2) a single transmembrane domain derived from the platelet-derived growth factor receptor, 3) an extracellular binding domain that contains four repeats of the titin Ig domain 127, which acts as a spacer, 4) A viral epitope that directs bait-PARCs towards patterns of cognate immobilized antibodies. The bait-PARCs, and the immobilized antibodies do not interact with cellular signal pathways and therefore minimally perturb cellular function. These bait-PARCs were used in experiments described herein below.

### Bait-PARCs Based on the Interaction Between Surface Immobilized DNA Oligonucleotides and DNA-Binding Protein Domains:

The bait-PARCs described in the section supra have the disadvantage that they require surface-immobilized antibody structures, which loose their functionality in denaturing conditions. To enable more flexible surface-modification technologies, zinc finger based artificial receptors that directly recognize DNA oligonucleotides were developed. DNA oligonucleotides have the advantage that they are both biologically stable and that they do not bind to endogenous cell surface receptors. Furthermore, a wide diversity of orthogonal pairs of DNA-binding artificial receptors and cognate oligonucleatides can be generated by creating permutations of known transcription activator-like effector (TALE) or zinc finger DNA-binding modules. Finally, DNA is a preferred extracellular compound for creating functionalized surfaces, as it is easily manipulated by a wide variety of linking techniques (Demers et al., 2002).

Here it is shown that zinc finger proteins can be used as artificial receptors to specifically recognize DNA oligonucleotides. As a proof of principle, the DNA binding section from the zinc-finger transcription factor Zif268 as the extracellular compound binding section, the transmembrane segment derived from the pDisplay vector (Invitrogen) and two fluorescent proteins—one intracellular and one extracellular—for localization of the receptors in living cells were implemented (FIGS. 3a and 3b). Any protein of interest can be linked as “bait” to the intracellular section for studying its interactions with a fluorescent “prey” in experiments analogous to the above. Two variants were constructed that either recognize the original Zif268 DNA sequence or a variant recognizing a triplet repeat of the central sequence portion. Both receptors specifically recognize and bind only their cognate DNA sequence and not the corresponding variant (FIG. 3b).

### Surface-Immobilized Molecules:

Biological or chemical compounds which interact with bait-PARCs are immobilized on a surface compatible with fluorescence-based measurements. Suitable molecules include, but are not limited to: growth factors, peptides or double-stranded DNA. Compounds are immobilized in structures of subcellular size by chemical or biological methods. Such methods include, but are not limited to: micrometer- or submicrometer scale surface patterns, micrometer- or submicrometer sized compound coated particles that can either be attached to a surface or freely moving with the cell. The following examples illustrate these possibilities:

### Micrometer Scale Patterns of Immobilized, Functional Antibodies Generated Via DNA-Directed Immobilization (DDI) and Dip Pen Nanolithography (DPN):

DNA-directed immobilization (DDI) (Niemeyer et al., 1994) was used to generate micrometer-scale arrays of antibodies with binding specificity for the peptide epitope on the bait-PARC. The DDI method takes advantage of specific hybridization of complementary oligonucleotides and thereby allows the site-specific capture of sensitive biomolecules at microstructures on a solid substrate under mild conditions. Furthermore, the DDI strategy allowed very flexible surface chemistry in the first microstructuring step, in which chemically stable capture-oligonucleotides were covalently linked to activated glass surfaces via dip-pen nanolithography (DPN) (Piner et al., 1999). Oligonucleotides complementary to the immobilized capture-oligonucleotides were covalently linked to streptavidin, and the resulting conjugates were functionalized with biotinylated antibodies and fluorophores. To generate a functional antibody array, these streptavidin-antibody complexes were then allowed to bind to the immobilized capture-oligonucleotide arrays. The high specificity of the interaction between distinct pairs of complementary DNA oligonucleotides enables the generation of multifunctional antibody arrays (FIG. 4a).

First, arrays of a single antibody were generated with average feature diameter of 4.5±0.5 μm and average feature distance of 11.4±1.4 μm (FIG. 4b). Bait-PARCs displaying 3 repeats of the VSVG epitope were recruited to anti-VSVG microstructures within the plasma membrane of living cells (265±55% enrichment of mean bait fluorescence intensity in comparison to non-targeted regions). Next miniaturized arrays of two distinct antibodies were generated via DDI. For this, two distinct capture-oligonucleotides were immobilized on glass surfaces in checkerboard patterns via DPN. Then these surfaces were incubated with a mixture of two complementary DNA-linked streptavidin conjugates labeled with spectrally separable biotinylated fluorophores. As shown in FIG. 4c, these two conjugates were selectively directed to their cognate microstructures. To limit the number of distinct fluorophores required to identify system components in subsequent experiments, antibody identity was encoded by fluorophore intensity. FIG. 4d shows checkerboard patterns of two distinct antibodies encoded by Atto 740 fluorescence intensity: anti-VSVG (high) and anti-HA (low). Bait-PARCs displaying either the VSVG or the HA epitope were enriched in their cognate antibody functionalized microstructures (289±125% mean bait fluorescence intensity for VSVG bait-PARCs (mTurquoise) and 322±127% for HA bait-PARCs (EGFP), compared to non-targeted regions). This shows that the spatially encoded information of an array of surface-linked antibodies can be transferred into an intracellular protein array via bait-PARCs.

Surface-Attached Submicrometer Scale Beads Coated with a Growth Factor:

Streptavidin-coated beads were functionalized with a biotin-linked fluorescent dye and biotin-linked EGF and immobilized on a biotin-functionalized glass surface (FIG. 5a). Cells expressing a kinase-dead fluorescently-labeled EGF-receptor show accumulation of the receptor to the location of EGF-coated beads (FIG. 5b). This method is technically simple, however, extending the number of extracellular compounds to generate an array requires fluorescent color-coding of the beads. Color-coding can limit the available fluorescent wavelengths for subsequent analysis (see below), however, as infra-red fluorescent dyes and suitable excitation and detection devices are now commonly available, at least 100 distinct fluorescent color combinations can be realized by using defined fluorescence intensities and ratios in mixtures of two different fluorescent dyes, which do not overlap with spectra of currently available fluorescent proteins typically used to label “bait” and “prey” proteins.

Internalized, Mobile Submicrometer Scale Beads Coated with Growth Factor:

Analogous to the above, streptavidin-coated beads were functionalized with a biotin-linked fluorescent dye and biotin-linked EGF. However, in this case, the linkage to the glass surface, on which cells grow, was weaker, allowing internalization of beads by cells. Imaging via standard fluorescence microscopy techniques allowed tracking of mobile, internalized submicrometer scale structures that accumulated the kinase-dead fluorescently labeled EGF-receptor in living cells (FIG. 5c). The structures were distinguishable from other cellular entities by their defined size and specific fluorescent properties. The advantage of such mobile beads is their ability to cover various spatial domains within a single cell over time. Integration of that activity can provide temporal averages of spatial activity distribution with a small number of particles.

### Surface Immobilization of Oligonucleotides Via Photolithography:

Glass coverslips were functionalized with olefin endgroups via amino-silane, PDITC, a 4th generation PAMAM dendrimer and the alkene-linker 1. Direct immobilization of thiol-functionalized oligos was induced by the light activated thiol-en reaction (Jonkheijm et al., 2008). Here, the 405 nm line of a scanning confocal microscope was used to directly write oligonucleotides on a glass surface (FIG. 6a). The immobilized, accessible single-stranded DNA was detected by hybridization with a fluorescently labeled complementary DNA oligonucleotide (FIG. 6b).

Extending the number of extracellular compounds to generate an array only requires sequential repetition of the photolithography process with different oligonucleotides. Such an array of oligonucleotides can be functionalized in a subsequent step via DNA-directed immobilization (Niemeyer et al., 1994) to immobilize any secondary compound such as a growth factor, an arbitrary peptide sequence or an antibody (FIG. 7). Such secondary compounds can be used to selectively recruit bait-PARCs that contain an extracellular cognate binding domain. Alternatively, double-stranded oligonucleotides arrays can also be used to directly and selectively recruit bait-PARCs based on TAL-repeats or zinc-finger domains. Analogous indirect linking strategies can be employed to functionalize beads.

### Direct Immobilization of Oligonucleotides Via Dip-Pen Nanolithography:

The micro-patterns of oligonucleatides shown in FIG. 4C were generated by dip-pen nanolithography (Salaita et al., 2007). Specifically, alkylamino-modified oligonucleotides were directly immobilized on epoxy-functionalized glass surfaces. This method can in principle be scaled down to very small feature dimensions (<50 nm).

### Fluorescent, Potential Intracellular Binding Partner, “Prey” Molecule:

Bait-PARCs are used in combination with a fluorescently labeled, potential intracellular binding partner, termed the “prey”. A wide variety of applications is possible with using just a single fluorescent label linked to one or more potential intracellular binding partners. However, in certain cases, if individual interaction pairs influence each other, multiple fluorescent colors, which can be distinguished by their excitation/emission spectra can be used. Potential intracellular binding partners include, but are not limited to molecules (biopolymers or small chemical agents), which are fluorescent by themselves, directly labeled with a fluorescent dye or genetically encoded fusion proteins of fluorescent proteins as in the following example.

Interaction Between the Regulatory and Catalytic Subunits of the Signal Protein PKA (cAMP-Dependent Protein Kinase):

Agonist induced activation of G-protein coupled receptors (GPCRs) leads to the dissociation of regulatory and catalytic subunits of the cAMP dependent protein kinase A (PKA) (Wong and Scott, 2004). This well-established signaling response was employed to validate our approach to study protein interactions in living cells via bait-PARCs. The regulatory subunit II-β (RII-β) was used as bait and fused to the intracellular region of bait-PARCs displaying VSVG epitopes (FIG. 8a). These RII-β presenting artificial receptor constructs were termed VSVG RII-β-PARC. The cytoplasmic catalytic subunit cat-α of PKA is fused to the fluorescent protein mCherry and acts as prey (mCherry-cat-α). As shown in FIG. 8b, the cytosolic prey protein was recruited to bait-PARC enriched microstructures in resting cells, which contain low levels of cAMP. Within seconds upon addition of the β-adrenergic receptor agonist isoproterenol, the interaction between bait and prey was lost (FIG. 8b). This shows that activation of these G-protein coupled receptors increases intracellular cAMP levels, which causes the dissociation of the catalytic subunits from regulatory subunits on bait-PARCs. Furthermore, direct and maximal elevation of intracellular cAMP levels by pharmacological stimulation of adenylate cyclase and inhibition of phosphodiesterase via forskolin/IBMX lead to a strong and persistent dissociation of the catalytic and regulatory subunits. This effect was fully reversible following drug washout.

To demonstrate that the dynamics of two distinct protein interactions can be monitored in single cells, two bait-PARCs were generated that were fused to bait proteins with distinct response properties: the regulatory subunits RI-α and RII-β. Each bait-PARC also displays distinct peptide antigens that are recognized by two corresponding, immobilized antibodies. The bait-PARCs were also fused to the spectrally separable fluorescent proteins mTurquoise and EGFP, respectively (FIG. 8a). As shown in FIG. 8c, the cytosolic prey protein mCherry-cat-α interacts with both bait-PARCs in resting cells. Normalization of prey recruitment to the enrichment of bait proteins allowed direct comparison of the cAMP dependent regulation of interactions between mCherry-cat-α and the regulatory subunits RI-α and RII-β in individual cells. This key feature enables the identification of relations between these distinct protein interactions. Indeed, it was found that mCherry-cat-α bound preferentially to RII-β in resting cells (FIGS. 8c and 9a).

Simultaneous monitoring of the response profiles of the two distinct bait-prey interactions also enabled analysis of their temporal correlation. In selected individual cells, a clear positive temporal cross-correlation for the cat-α/RI-α and the cat-α/RII-β interaction responses to β-adrenergic receptor stimulation was observed. However, the average cross-correlation from several cells was much weaker (FIG. 9b). Treatment with IBMX/forskolin always strongly and reversibly dissociated the interaction between the catalytic and both regulatory PKA subunits, demonstrating their intact functionality (FIG. 9c). In comparison to this pharmacological effect, only a subset of cells responded to β-adrenergic receptor stimulation (FIG. 8c). This high level of cell-to-cell variance can be explained by adaptive mechanisms in the underlying signal networks. Due to this variance, relations in response properties between the regulatory subunits, such as their interaction efficiency in resting cells (FIG. 9a) or their temporal cross-correlation profiles (FIG. 9b), are blurred in averaged measurements from many cells. This is overcome by measuring the dynamic response profiles of the interaction between the catalytic and the two distinct regulatory subunits simultaneously in individual cells.

### Example 3

### Applications of the Present Invention

### Measurements of Single or Multiple Protein Interactions or Protein Reactions in an Individual Cell

Protein interactions play a pivotal role in cellular regulation both in physiological as well as pathophysiological conditions. Dynamic changes in protein interactions are indicative of their activity state and can thus be used to quantify biological processes at a level of molecular detail. While the measurement of an individual protein interaction or protein activity can be highly informative, many fundamental cellular processes, such as the determination of cell growth vs. cell shape changes are encoded by combinations of multiple dynamic activities. Available methods for measuring the dynamics of protein interactions in individual, living cells are limited to one—or in highly specialized experiments—a few activities at most. The described invention is enabling simultaneous measurements of multiple activities. The general implementation of this invention and the scalable concept of bait-PARCs based on antibody-epitope interactions or zinc-finger/DNA interactions offers a way to generate live cell multiplex biosensors to follow many protein interactions at the same time in an individual cell. The following concepts are, for example, possible:

### Multiplex Sensors for Known, Orthogonal Protein Interaction Pairs

The immobilized “bait” proteins that are linked via bait-PARCs are easily distinguished via spectral properties of functionalized beads or their relative positioning on a modified surface. The intracellular “prey”, on the other hand has to be identified via its fluorescent color—which is limited to a few spectral variants. However, if the measurements are limited to orthogonal interactions (e.g. no cross talk—no cross modulation between interaction pairs), multiple intracellular sensor proteins can be labeled with the same fluorescent protein or dye, as only one prey molecule would be able to interact with it's specific cognate bait-PARC. Examples of this type of multiplex biosensor could be composed of artificial receptors, which use the GTPases Ras, RhoA and cdc42 as “bait”, which interact—in an activity dependent manner—specifically only with their cognate, activity-dependent binding domains derived from Raf-kinase, rhotekin and N-Wasp, respectively.

Such multiplex biosensors of orthogonal activity detection pairs can yield highly detailed information on the dynamics of interrelated signal activities. For example, by analyzing the combinatorial dynamics of such activities, in combination with acute perturbations, the dynamic interplay between individual components can be analyzed. The analysis of such interplay is not limited to the analysis of temporal dynamics: By using artificial receptors on mobile beads as sensors, or by generating repetitive arrays of few selected activities, the spatial distribution of activities can also be mapped within individual cells.

### Multiplex Biosensors for the De-Novo Discovery and Dynamic Analysis of Protein Interactions:

Live cell multiplex biosensors can also be used to identify new protein interactions. For example, a single protein of interest can be linked to a fluorescent protein as the intracellular “prey” component and then be probed against a panel of immobilized “bait” candidate interaction partners. In comparison to established techniques, such as yeast two-hybrid or mass spectrometry approaches, this invention allows the identification of novel protein interactions in intact living cells in the natural context of the protein of interest. While the mass spectrometry approaches can address protein interactions in their natural context, the method requires the destruction of the cells. On the other hand, the yeast two-hybrid system allows the identification of protein interactions in living cells, but it's biological context is limited to the nucleoplasm of yeast cells and therefore not a natural context for many applications.

The main advantage of this invention in the context of identifying novel protein interactions is, however, the ability to dynamically manipulate the cellular context during the experiment. For example, a protein interaction might only be relevant during a particular dynamic state of the cell after hormonal cell stimulation or during a particular stage of the mitotic cycle. As this invention can be applied to the identification of new protein interactions in individual, living, intact cells, such dynamic, transient interactions, which might be elusive in other, standard methods, can be accessible via this invention.

### Analysis of the Cytoplasmic State of Individual Cells:

The behavior of individual cells is usually not defined by a single biological activity, but instead directed by a combinatorial set of activities, here denoted as a cytoplasmic state (Niethammer et al. 2007). As a larger cell population usually contains cells with different momentary behaviors, the combination of such activities is very different between individual cells. In an analysis of the whole population, such differences will average out, leading to a readout of signals, that is not representative of the original activity combinations in individual cells.

Via the ability to study multiple protein interactions in individual living cells, this invention offers a way to determine the cytoplasmic state of individual cells. As the simplest example, one or more central signal molecules that form central nodes in interaction networks can be linked to fluorescent proteins and serve as intracellular “prey”. Their interactions with many known interaction partners can then be measured simultaneously via live cell multiplex biosensors.

Our current knowledge about cytoplasmic states is very limited, as the few known examples required decades of laborious work to identify the individual interactions, their causal dependencies and their biological meaning. This invention can speed up this process by orders of magnitude, as it allows—for the first time—a straightforward and direct measurement of the real-time dynamics of cytoplasmic states. Direct correlation of cell behavior—in unstimulated or stimulated conditions—with the measurements of cytoplasmic states defined by key regulatory signaling node interaction maps, will allow the rapid identification of behavior specific cytoplasmic states of individual cells.

### Development of Cell-Based Sensors for Clinical and Environmental Applications:

The knowledge that can be derived from measuring multiple protein interactions in applications described above—and especially the correlation of cell behavior, with quantifiable cytoplasmic states will allow the development of cell based sensors for compounds that induce changes in the cytoplasmic state. Medically relevant cytoplasmic states include for example apoptosis, necrosis, proliferation, transformation, senescence, differentiation, cell growth, cell shrinkage, etc. Detection devices that are based on such sensors include, but are not limited to: analysis of growth factors in medical samples or analysis of toxic test compounds.

1. A method for determining whether a reaction occurs between a chimeric transmembrane receptor and an intracellular interaction partner thereof within a cell, said method comprising the steps of:
a. providing a cell comprising:
i. at least two distinct chimeric transmembrane receptors each comprising:
(a) an extracellular binding domain,
(b) a transmembrane domain, and
(c) an intracellular domain,
wherein said at least two transmembrane receptors are distinct in that (i) at least two of the domains (a), (b) and (c) are of different origin, (ii′) in that said extracellular binding domain of each of said at least two transmembrane receptors specifically interacts with a different extracellular compound, and (iii′) in that said intracellular domain of each of said at least two transmembrane receptors is different; and
ii. one or more different potential intracellular interaction partners that (i′) for step b.i. or step b.ii.(i′) or (ii′) are labelled with first labels; or (ii′) for step b.ii.(iii′) are unlabelled;
b. contacting the cell with at least two different extracellular compounds, wherein each of said at least two extracellular compounds is bound to a surface
i. in different areas of the same support, and/or
ii. on different supports, (i′) wherein each support and its cognate transmembrane receptor form a complex that is labelled with a second label, (ii′) wherein each support can be distinguished by its shape and/or size, and/or (iii′) wherein in each support and its cognate transmembrane receptor the intracellular domain is labelled with a label or a pair of labels which is capable to indicate reactions with one or more potential intracellular interaction partners of step a.ii. with the intracellular domain; and
c. detecting
i. said first label in step b.i.; and/or
ii. said first label and second label, shape and/or size in step b.ii.; wherein
i for step c.i. the presence of a signal of said first label in (an) area(s) comprising the cognate extracellular compound and
ii for step c.ii.(i′) the presence of co-localized signals of said first and second label(s), for step c.ii.(ii′) co-localization of said first signal with said support, and for step c.ii.(iii′) a detectable conformational change of the label or a detectable energy transfer between the pair of labels
is indicative of a reaction between a potential intracellular interaction partner with a distinct chimeric transmembrane receptor.

2. The method of claim 1, wherein the reaction is an interaction between a chimeric transmembrane receptor and an intracellular interaction partner or a protein conformational change of the chimeric transmembrane receptor and/or the intracellular interaction partner.

3. The method of claim 1, wherein the reaction is selected from the group consisting of phosphorylation, glycosylation, lipidation (such as myristoylation, palmitoylation, prenylation), proteolytic cleavage, acetylation, disulfide bond formation, alkylation (such as methylation), ubiquitination, SUMOylation, oxidation, nitrosylation, nucleotide addition (such as ADP-ribosylation), adenylylation, arginylation, racemization of proline and the corresponding reverse reactions of the before listed reactions.

4. The method of claim 2, wherein the reaction between a chimeric transmembrane receptor and an intracellular interaction partner thereof is an interaction between a chimeric transmembrane receptor and an intracellular interaction partner thereof, whereby the intracellular interaction partner is an intracellular binding partner.

5. The method of claim 1, wherein detection of said first and/or said first and second labels, shape and/or size in step c. is effected over a period of time continuously or intermittently, thereby monitoring said reaction between said potential intracellular interaction partner(s) and said at least two transmembrane receptors.

6. The method of claim 1, wherein the determining involves a quantification of said reaction(s) between said potential intracellular interaction partner(s) and said at least two transmembrane receptors.

7. The method of claim 1, wherein at least one of said at least two different extracellular compounds is bound to the surface of said same support more than once and in different areas to be covered by said cell; or wherein each of said at least two different extracellular compounds is bound to more than one of said different supports.

8. The method of claim 1, wherein the cell comprises at least two different potential intracellular interaction partners and wherein the intracellular domains of each of said at least two transmembrane receptors specifically interact with one of said at least two different potential intracellular interaction partners, respectively.

9. The method of claim 1, wherein said different extracellular compounds do not specifically interact with an endogenous transmembrane receptor of the cell provided in step a.

10. The method of claim 1, wherein said surface to which said different extracellular compounds are bound is a planar or a spherical surface.

11. The method of claim 1, wherein said different supports in step b.ii. can be taken up by said cell provided in step a.

12. The method of claim 1, wherein said domains (a), (b) and (c) are synthetically designed domains or domains obtained from at least two different proteins of one or more species.

13. The method of claim 1, wherein said extracellular binding domain and said transmembrane domain are inert with regard to triggering an intracellular response.

14. The method of claim 1, wherein said extracellular binding domain is a protein binding domain, an antibody epitope, an antibody, an oligonucleotide binding domain, or a small molecule binding domain.

15. The method of claim 14, wherein said oligonucleotide binding domain comprises or consists of one or more zinc finger domains, TAL repeats, helix-turn-helix domains, leucine zippers, winged helix domains, winged helix-turn-helix domains, helix-loop-helix domains, HMG-boxes, (mutant) restriction nucleases, PUF repeats, zinc-containing RNA binders, KH domains, RRM domains or RBD/RRM/RNP domains.

16. The method of claim 1, wherein (i) said extracellular binding domain and said transmembrane domain and/or (ii) said transmembrane domain and said intracellular domain are connected by a linker sequence, said linker being preferably one or more (biologically inert) immunoglobulin domains, a flexible domain peptide linker, such as a Glycine-Serine linker, or a rigid linker, such as a helix-forming rigid linker.

17. The method of claim 1, wherein said transmembrane domain is an artificially designed transmembrane domain, an alpha-helical transmembrane domain of a single-span membrane protein, a transmembrane domain of a growth factor receptor, or a multiple-pass transmembrane domain.

18. The method of any one of claims 1 to 17, wherein the intracellular domain comprises or consists of a protein binding domain, a small molecule binding domain, a oligonucleotide binding domain, or a sensor construct, an enzyme, an antibody epitope, a chelator.

19. A kit comprising:
a. (i) a cell comprising:
i. at least two distinct chimeric transmembrane receptors each comprising:
(a) an extracellular binding domain,
(b) a transmembrane domain, and
(c) an intracellular domain,
wherein said at least two transmembrane receptors are distinct in that (i) at least two of the domains (a), (b) and (c) are of different origin, (ii′) in that said extracellular binding domain of each of said at least two transmembrane receptors specifically interacts with a different extracellular compound, and (iii′) in that said intracellular domain of each of said at least two transmembrane receptors is different; and
ii. one or more different potential intracellular interaction partners that (i′) for element (ii) i. or element (ii) ii.(i′) or (ii′) are labelled with first labels; or (ii′) for element (ii) ii.(iii′) are unlabelled, and
(ii) at least two different extracellular compounds, wherein each of said at least two extracellular compounds is bound to a surface
i. in different areas of the same support, and/or
ii. on different supports, (i′) wherein each support and its cognate transmembrane receptor form a complex that is labelled with a second label, (ii′) wherein each support can be distinguished by its shape and/or size, and/or (iii′) wherein in each support and its cognate transmembrane receptor the intracellular domain is labelled with a label or a pair of labels which is capable to indicate reactions with one or more potential intracellular interaction partners of element a. (i) ii. with the intracellular domain; and/or
b. (i) at least two different extracellular compounds, wherein each of said at least two extracellular compounds is bound to a surface
i. in different areas of the same support, and/or
ii. on different supports, (i′) wherein each support and its cognate transmembrane receptor encoded by nucleic acid molecules of element b.(ii) form a complex that is labelled with a second label, (ii′) wherein each support can be distinguished by its shape and/or size, and/or (iii′) wherein in each support and its cognate transmembrane receptor encoded by nucleic acid molecules of element b.(ii) the intracellular domain is labelled with a label or a pair of labels which is capable to indicate reactions with one or more potential intracellular interaction partners encoded by nucleic acid molecules of element
b.(ii) with the intracellular domain; and
(ii) nucleic acid molecules encoding the one or more potential intracellular interaction partners as defined in element a.(i) ii. above and the at least two distinct chimeric transmembrane receptors as defined in element a.(i) i. above.

20. A chimeric transmembrane receptor, comprising:
(a) an extracellular binding domain comprising an epitope which is connected to the transmembrane domain of (b) via four repeats of the titin Ig domain 127,
(b) a transmembrane domain comprising a single transmembrane domain obtained from the platelet-derived growth factor receptor, and
(c) an intracellular domain which comprises (i) a protein of interest and (ii) a fluorescent protein.
